(19) United States
(12) Patent Application Publication
(10) Pub. No.: US 2013/0084272 A1
(43) Pub. Date: Apr. 4, 2013

(54) COMPOSITIONS, KITS AND METHODS FOR NUTRITIONAL SUPPLEMENTATION WITH TWELVE CARBON CHAIN FATTY ACIDS AND TWELVE CARBON CHAIN ACYLGLYCEROLS

(71) Applicants: Philippe PERRIN, Paris (FR); Guillaume HERRY, Hoboken, NJ (US); Cecile BOYER-JOUBERT, Fontenay Aux Roses (FR)

(72) Inventors: Philippe PERRIN, Paris (FR); Guillaume HERRY, Hoboken, NJ (US); Cecile BOYER-JOUBERT, Fontenay Aux Roses (FR)

(21) Appl. No.: 13/248,851

(22) Filed: Sep. 29, 2011

(51) Int. Cl. A61K 38/43; A61K 31/714; A61K 31/519; A61K 31/59; A61K 33/18; A61K 31/20; A61K 31/202; A61K 31/51; A61K 31/455; A61K 31/375; A61K 33/32; A61K 33/34; A61K 33/06; A61K 31/4188; A61K 31/355; A61K 33/00; A61K 33/04; A61K 33/42; A61P 39/00; A61K 33/26

(52) U.S. Cl. 424/94.1; 424/646; 514/52; 514/252.16; 514/167; 424/667; 514/558; 514/560; 514/276; 514/356; 514/474; 424/641; 424/630; 424/682; 514/387; 514/458; 424/661; 424/639; 424/702; 424/601

(57) ABSTRACT

The present invention relates to compositions, kits and methods for the administration of compositions and kits for supplementation with twelve carbon chain fatty acids or twelve carbon chain acylglycerols; and specifically for the supplementation with twelve carbon chain fatty acids such as lauric acid and twelve carbon chain acylglycerols such as monolaurin; and specifically for the co-supplementation with other vitamins, minerals and nutrients.

### FIELD OF THE INVENTION

The present invention relates to various compositions and kits and methods of administration of compositions and kits for nutritional supplementation with twelve carbon chain fatty acids such as lauric acid and twelve carbon chain acylglycerols such as monolaurin.

### BACKGROUND OF THE INVENTION

The human consumption of fats and oils has changed substantially over the last half century. In particular, diets have been shown to substantially increase in the consumption of partially hydrogenated oils and trans fats. The consumption of trans fats has been a well-known factor for the increased risk of coronary heart disease in the United States. For example, it has been well documented that the fast-food industry has used partially hydrogenated oils for frying. This drastic change in diet has resulted in the decreased consumption of other oils and fats, many of which have multiple health benefits. Particularly, there has been a decreased consumption of healthy oils and fats from certain fish and plants. Coconut oil, for example, is rich in medium-chain fatty acids, such as twelve carbon chain fatty acids, many of which have multiple health benefits. Indeed, coconut oil contains about 50% of the twelve carbon chain fatty acid lauric acid, which possesses many antibacterial, antiviral, and antifungal properties and thus has an overall anti-infectious benefit. dela Paz et al., PHILLIPINE JOURNAL OF INTERNAL MEDICINE, The Effect of Virgin Coconut Oil on Lipid Profile and Fasting Blood Sugar: A Phase I Clinical Trial, 48(2); 1-7 (2010).

Acylglycerols, which contain one to three fatty acid chains covalently bonded to a glycerol by an ester bond, can be precursors or metabolites of fatty acids that may provide similar benefits. For example, monolaurin, a metabolite of lauric acid, possesses antimicrobrial, antiviral, and antifungal properties, with apparently greater activity than lauric acid. Lieberman et al., ALTERNATIVE & COMPLEMENTARY THERAPIES, 310-314 (December, 2006); Batovska et al., POL. J. MICROBIOL. 58(1): 43-47 (2009).

In addition, a diet for prenatal, pregnant or lactating women that includes twelve carbon chain fatty acids or twelve carbon chain acylglycerols such as lauric acid or monolaurin translates into more of these compounds in mothers milk and thus may provide the overall anti-infectious benefits to an infant. Francois et al., AM J CLIN NUTR, 67(2): 301-8 (1998). Even further, there is evidence that a diet including twelve carbon chain fatty acids or twelve carbon chain acylglycerols heavily influences its blood serum levels in a patient; accordingly, these benefits may be provided to the fetus when provided in a pregnant women's diet. dela Paz et al., PHILLIPINE JOURNAL OF INTERNAL MEDICINE, The Effect of Virgin Coconut Oil on Lipid Profile and Fasting Blood Sugar: A Phase I Clinical Trial, 48(2); 1-7 (2010). Indeed, diet and supplementation with the appropriate nutrients is especially pertinent to women contemplating conceiving a child because optimizing specific nutrients before, during, and after the physiological processes of pregnancy or lactation can have profound, positive, and comprehensive impacts upon the overall wellness of the developing and newborn child as well as on the safety and health of the mother.

Because of these changes in diet, the numerous benefits of twelve carbon chain fatty acids or twelve carbon chain acylglycerols is presently deficient. Supplementation of the diet with these beneficial fatty acids and acylglycerols is thus currently needed. Such exogenous supplementation of these compounds, either alone or in a multivitamin nutritional supplement would be beneficial as there is no current multivitamin nutritional supplement that provides for an exogenous supplementation of these beneficial fatty acids and acylglycerols, for prenatal, pregnant and lactating women.

Other fatty acids deficient in the diet are omega-3 fatty acids such as DHA—which are prevalent in fish. DHA plays an integral role in physiological mechanisms that serve to prevent, treat and/or alleviate the occurrence or negative effects of some diseases.

DHA has shown multiple health-promoting properties in adults. These include anti-thrombotic, anti-inflammatory and anti-atherosclerotic activity, all of which reduce the risk of heart disease. M Laidlaw and B J Holub, AM J CLIN NUTR, 77:37-42 (2003). Inverse relationships have also been found between systemic levels of DHA and the incidence and severity of mood disorders and depression, including post-partum depression. Id. Therefore, introduction of omega-3 fatty acids such as DHA during pregnancy benefits both child and mother.

Accordingly, a nutritional supplement or multivitamin that includes twelve carbon chain fatty acids and/or twelve carbon chain acylglycerols with other vitamins, minerals and nutrients, such as DHA, would be particularly beneficial.

### SUMMARY OF THE INVENTION

The present invention provides compositions, kits and methods of administering compositions and kits for both prophylactic and therapeutic nutritional supplementation. Specifically, for example, the present invention relates to novel compositions and kits and methods of administration of compositions and kits for supplementation with twelve carbon chain fatty acids such as lauric acid and twelve carbon chain acylglycerols such as monolaurin.

In a specific embodiment of the present invention, the compositions may include a twelve carbon chain fatty acid or twelve carbon chain acylglycerol, omega-3 fatty acids, one or more vitamins, nutrients or minerals and one or more pharmaceutically acceptable carriers. In a specific embodiment, the twelve carbon chain fatty acid or twelve carbon chain acylglycerol comprises one or more of the group consisting of lauric acid, glycerol monolaurate (monolaurin), dilaurin, trilaurin, a twelve carbon chain monoacylglycerol, a twelve carbon chain unsaturated fatty acid, a diacylglycerol comprising at least one twelve carbon chain, and a triacylglycerol comprising at least one twelve carbon chain. In another embodiment, the twelve carbon chain fatty acid or twelve carbon chain acylglycerol may comprise one or more selected from the group consisting of lauric acid and monolaurin.

In another specific embodiment, the twelve carbon chain fatty acid or twelve carbon chain acylglycerol may be derived from a natural source, synthesized, or genetically engineered. In another embodiment, the natural source of the twelve carbon chain fatty acid or twelve carbon chain acylglycerol may be selected from one or more of the group consisting of algae oil, coconut oil, babassu palm oil, palm kernel oil, ouricuri oil, tucum oil, muru-muru oil, jaboty kernel oil, khakan kernel oil, dika nut oil, ucuhuba butter, cinnamon oil, Brachyandra Calophylla seed oil, California laurel seed oil, camphor kernel fat, cohune nut oil (Palm Oil), Cuphea seed oil, Heteranthus epilobiifolia seed oil, Irvingia Gabonesis Kernel Fat (Dika Fat), jack bean oil, Khakan fat, Pelu fat, Laurel Berry (Bay Berry) oil, Lindera Umbellata seed oil, Ouricouri Tallow, Pindo Palm kernel oil, Pisa oil, Spicebush Kernel fat, Tucum (Aoiara) Kernel oil, cuphea plants, Lauraceae plants, Umbelliferae plants (Apiaceae plants), Arecaceae palms, Lythraceae plants, fruits of Laurus nobilis, qualea plants, Actinodaphne seed oil, cohune palms (Attalea cohune, rain tree, american oil palm, corozo palm or manaca palm), Pycanthus kombo (african nutmeg), Virola surinamensis (wild nutmeg), peach palm seed, betel nut, date palm seed, macadamia nut, plum, Watermelon seed, Citrullus lanatus (egusi melon), and Pumpkin flower. In a specific embodiment, the natural source of the twelve carbon chain fatty acid or twelve carbon chain acylglycerol may be selected from one or more of the group consisting of coconut oil, palm kernel oil, babassu oil and algae oil.

In another specific embodiment, the twelve carbon chain fatty acid or twelve carbon chain acylglycerol may be present in an amount greater than about 20 mg. In another embodiment, the twelve carbon chain fatty acid or twelve carbon chain acylglycerol may be present in an amount of about 30 mg to about 10 g. In another embodiment, the twelve carbon chain fatty acid or twelve carbon chain acylglycerol may be in the form of lauric acid and may be present in an amount greater than about 20 mg, from about 30 mg to about 10 g, or about 30 mg to about 280 mg. In another specific embodiment, the twelve carbon chain fatty acid or twelve carbon chain acylglycerol may be in the form of lauric acid and may be present in an amount of about 60 mg.

In another embodiment of the present invention, the vitamins, nutrients or minerals may be selected from one or more of the group consisting of vitamin D, iodine, vitamin B9, vitamin B 12, and iron. In a specific embodiment, vitamin D, if present, may be in an amount of about 200 I.U. to about 1600 I.U., iodine, if present, may be in an amount of about 100 μg to about 300 μg, vitamin B9, if present, may be in an amount of about 0.2 mg to about 1.5 mg, vitamin B12, if present, may be in an amount of about 2 μg to about 18 μg, and iron, if present, may be in an amount of about 13.5 mg to about 40.5 mg.

In another embodiment, the vitamins, nutrients or minerals may be selected from one or more of the group consisting of vitamin D, iodine, vitamin B1, vitamin B2, vitamin B3, vitamin B6, vitamin B9, vitamin B12, vitamin E, vitamin A, vitamin C, iron, zinc, copper, and magnesium. In another embodiment of the present invention, the vitamins, nutrients or minerals may be selected from one or more of the group consisting of vitamin D, iodine, vitamin B1, vitamin B2, vitamin B3, vitamin B6, vitamin B7, vitamin B9, vitamin B12, vitamin E, vitamin A, vitamin C, iron, zinc, copper, magnesium, calcium, molybdenum, chlorine, vitamin K, manganese, selenium, choline, phosphorous, bioflavonoids, CoQ₁₀, chromium, alpha lipoic acid, lutein and antioxidants.

In another embodiment, omega 3 fatty acids may be in the form selected from one or more of the group consisting of docosahexaenoic acid (DHA), eicosapentaenoic acid (EPA) and α-linolenic acid (ALA). In a specific embodiment, the omega 3 fatty acids may comprise DHA. In a specific embodiment the source of DHA may be from one or more of the group consisting of animal, fish, plants, algae, microorganism production or production by genetic engineering. In a specific embodiment, the source of DHA may be fish oil. In another specific embodiment, the source of DHA may be algae oil.

In a specific embodiment of the compositions of the present invention, omega three fatty acids such as DHA may be present in various amounts and ranges. In another embodiment, the DHA may be present in an amount of about 100 mg to about 300 mg. In another embodiment, the DHA may be present in an amount of about 180 mg to about 220 mg. In another embodiment, the DHA may be present in an amount of about 200 mg.

In another specific embodiment of the compositions of the present invention, twelve carbon chain fatty acid or twelve carbon chain acylglycerols such a lauric acid and monolaurin may be provided from various sources. In a specific embodiment, the source of lauric acid and/or monolaurin may be selected from one or more of the group consisting of fish oil, algae oil and coconut oil. In a specific embodiment, the sources may be algae oil and coconut oil. In another specific embodiment, lauric acid and/or monolaurin may be present in an amount of about 5 mg to about 30 mg from algae oil and about 25 mg to about 250 mg from coconut oil. In another specific embodiment, lauric acid and/or monolaurin may be present in an amount of about 5 mg to about 25 mg from algae oil and about 25 mg to about 100 mg from coconut oil. In another specific embodiment, lauric acid and/or monolaurin may be present in an amount of about 10 mg from algae oil and about 50 mg from coconut oil. In another specific embodiment, lauric acid and/or monolaurin may be present in an amount of about 5 mg to about 30 mg from algae oil and about 25 mg to about 250 mg from coconut oil and omega 3 fatty acids in the form of DHA may be present in an amount of about 100 mg to about 300 mg. In another specific embodiment, lauric acid and/or monolaurin may be present in an amount of about 10 mg from algae oil and 50 mg from coconut oil and DHA may be present in amount of about 200 mg. In another specific embodiment, the coconut oil may be present in an amount of about 50 mg to about 500 mg and algae oil may be present in an amount of about 200 mg to about 600 mg.

In another specific embodiment of the present invention, the compositions may be a dietary supplement. In another embodiment, the compositions may be a prenatal supplement. In another embodiment, the compositions may be a supplement for women during pregnancy. In another embodiment, the compositions may be a postnatal supplement. In another embodiment, these compositions for pregnancy, prenatal and postnatal supplementation may be for prescription only. In another embodiment, these compositions may be a prescription drug.

In another embodiment, the compositions of the present invention may be provided in various dosage forms. In a specific embodiment, the dosage form may be selected from one or more of the group consisting of a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution. In a specific embodiment, the dosage form may be a gelcap.

In another embodiment of the present invention, the methods may include administering the compositions of the present invention to a patient. In a specific embodiment, the patient may be in need of twelve carbon chain fatty acid or twelve carbon chain acylglycerol supplementation or the supplementation may be for prophylactic purposes. In another specific embodiment, the patient may be in need of twelve carbon chain fatty acid or twelve carbon chain acylglycerols as an antiviral, a bactericidal, an antifungal or as a general antimicrobial. In another specific embodiment, the patient may be human. In another embodiment, the human may be a women. In another embodiment, the woman may be pregnant, prenatal or breast feeding.

In another embodiment of the present invention, the compositions may be provided in dosing volumes, for example greater than about 50 ml. In another embodiment, the dosing volumes may be greater than 100 ml. In another embodiment, the dosing volume may be greater than 500 ml. In another specific embodiment, the dosing volume may be in range of about 100 ml to about 500 ml. In another embodiment, the dosing volume may be in a range of about 100 ml to about 300 ml. In a specific embodiment, the compositions may include a twelve carbon chain fatty acid or twelve carbon chain acylglycerol, one or more vitamins, nutrients or minerals and one or more pharmaceutically acceptable carriers, wherein the composition comprises a dosing volume greater than about 50 ml. In a specific embodiment, the twelve carbon chain fatty acid or twelve carbon chain acylglycerol comprises one or more of the group consisting of lauric acid, glycerol monolaurate (monolaurin), dilaurin, trilaurin, a twelve carbon chain monoacylglycerol, a twelve carbon chain unsaturated fatty acid, a diacylglycerol comprising at least one twelve carbon chain and a triacylglycerol comprising at least one twelve carbon chain. In another specific embodiment, the twelve carbon chain fatty acid or twelve carbon chain acylglycerol comprises one or more of the group consisting of lauric acid and monolaurin. In another specific embodiment, the twelve carbon chain fatty acid or twelve carbon chain acylglycerol may be present in an amount greater than about 20 mg. In a specific embodiment, the twelve carbon chain fatty acid or twelve carbon chain acylglycerol may be in the form selected from the group consisting of lauric acid and monolaurin and may be present in amount of about 0.1 g to about 2.4 g.

In another specific embodiment, the compositions may comprise a dosing volume greater than about 50 ml in a dosage form selected from the group consisting of a syrup, a liquid composition, a powder, a concentrated powder, a concentrated powder admixed with a liquid, a chewable form, a swallowable form, a dissolvable form, an effervescent, and an oral liquid solution. In a specific embodiment the dosage form may be a liquid composition. In another specific embodiment, the dosage form may be in a concentrated powder or a concentrated powder that may be admixed with a liquid for a dosing volume greater than about 50 ml. In another specific embodiment, the twelve carbon chain fatty acid or twelve carbon chain acylglycerol may be from a natural source, may be synthesized, or may be derived from a genetically modified or engineered source. In another specific embodiment, the natural source of the twelve carbon chain fatty acid or twelve carbon chain acylglycerol may be selected from one or more of the group consisting of algae oil, Martek algae oil, coconut oil, babassu palm oil, palm kernel oil, ouricuri oil, tucum oil, muru-muru oil, jaboty kernel oil, khakan kernel oil, dika nut oil, ucuhuba butter, cinnamon oil, Brachyandra Calophylla seed oil, California laurel seed oil, camphor kernel fat, coconut oil, cohune nut oil (Palm Oil), Cuphea seed oil, Heteranthus epilobiifolia seed oil, Irvingia Gabonesis Kernel Fat (Dika Fat), jack bean oil, Khakan fat, Pelu fat, Laurel Berry (Bay Berry) oil, Lindera Umbellata seed oil, Ouricouri Tallow, palm kernel oil, Pindo Palm kernel oil, Pisa oil, Spicebush Kernel fat, Tucum (Aoiara) Kernel oil, cuphea plants, Lauraceae plants, Umbelliferae plants (aka Apiaceae plants), Arecaceae palms, Lythraceae plants, fruits of Laurus nobilis, qualea plants, Actinodaphne seed oil, cohune palms (also known as Attalea cohune, rain tree, american oil palm, corozo palm or manaca palm), Pycanthus kombo (also known as african nutmeg), Virola surinamensis (aka Wild nutmeg), peach palm seed, betel nut, date palm seed, macadamia nut, plum, Watermelon seed, Citrullus lanatus (egusi melon), and Pumpkin flower. In another specific embodiment, the natural source of the twelve carbon chain fatty acid or twelve carbon chain acylglycerol may be selected from one or more of the group consisting of coconut oil, palm kernel oil, babassu oil and algae oil. In a specific embodiment, the twelve carbon chain fatty acid or twelve carbon chain acylglycerol may be selected from the group consisting of lauric acid and monolaurin and the source of said lauric acid and monolaurin may be coconut oil and/or algae oil.

In another specific embodiment of the compositions comprising a dosing volume greater than about 50 ml, the one or more vitamins, nutrients or minerals may comprise omega 3 fatty acids. In a specific embodiment, the omega 3 fatty acids may be in the form selected from one or more of the group consisting docosahexaenoic acid (DHA), eicosapentaenoic acid (EPA) and α-linolenic acid (ALA). In a specific embodiment, the omega 3 fatty acids may comprise DHA. In another specific embodiment, the source of DHA may comprise one or more of the group consisting of animal, fish, plants, algae or microorganism production. In a specific embodiment, the source of DHA may be fish oil. In another specific embodiment, the source of DHA may be algae oil. In another embodiment, the DHA may be present in an amount of about 100 mg to about 300 mg. In another embodiment, the DHA may be present in an amount of about 180 mg to about 220 mg. In a specific embodiment, the DHA may be present in an amount of about 200 mg.

In another embodiment, the compositions comprising a dosing volume greater than about 50 ml may include vitamins, nutrients or minerals selected from one or more of the group consisting of vitamin D, iodine, vitamin B1, vitamin B2, vitamin B3, vitamin B6, vitamin B9, vitamin B12, vitamin E, vitamin A, vitamin C, iron, zinc, copper, magnesium, and omega 3 fatty acids. In another embodiment, the vitamins, nutrients or minerals may be selected from one or more of the group consisting of vitamin D, iodine, vitamin B1, vitamin B2, vitamin B3, vitamin B6, vitamin B7, vitamin B9, vitamin B12, vitamin E, vitamin A, vitamin C, iron, zinc, copper, magnesium, omega 3 fatty acids, calcium, molybdenum, chlorine, vitamin K, manganese, selenium, choline, phosphorous, bioflavonoids, CoQ₁₀, chromium, alpha lipoic acid, choline, lutein and antioxidants.

The compositions comprising a dosing volume greater than about 50 ml may include additional components, such as milk protein concentrate. In a specific embodiment, the compositions may further comprise one or more ingredients selected from the group consisting of water, sucrose, maltodextrin, milk protein concentrate, soy oil, canola oil, short chain fructooligosaccarides, soy protein isolate, corn syrup, sodium caseinate, and potassium citrate. In another embodiment, the composition may comprise a natural or artificial flavor selected from one or more of the group consisting of the flavors apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, and watermelon. In another specific embodiment of the present invention, the compositions may be a dietary supplement. In another embodiment, the compositions may be a prescription prenatal supplement. In another embodiment, the compositions may be a prescription supplement for women during pregnancy. In another embodiment, the compositions may be a prescription postnatal supplement. In another embodiment, the compositions may be a prescription drug.

In another embodiment of the invention, the compositions comprising a dosing volume greater than about 50 ml may be administered to a patient. In a specific embodiment, the patient may be in need of twelve carbon chain fatty acid or twelve carbon chain acylglycerol supplementation. In another specific embodiment, the patient may be in need of twelve carbon chain fatty acid or twelve carbon chain acylglycerols as an antiviral, a bactericidal, an antifungal or one or more combinations thereof. In another specific embodiment, the patient may be human. In another embodiment, the human may be a women. In another embodiment, the woman may be pregnant, prenatal or breast feeding.

One embodiment of the present invention may include multiple compositions comprising a twelve carbon chain fatty acid or twelve carbon chain acylglycerol, an omega 3 fatty acid, and one or more vitamins, minerals and nutrients, provided in a kit. In a specific embodiment, the kits of the present invention may comprise a first composition comprising one or more selected from the group consisting of twelve carbon chain fatty acids, twelve carbon chain acylglycerols, omega 3 fatty acids, vitamins, nutrients, minerals, and optionally one or more pharmaceutically acceptable carriers; and a second composition comprising one or more selected from the group consisting of twelve carbon chain fatty acids, twelve carbon chain acylglycerols, omega 3 fatty acids, vitamins, nutrients, and minerals, and one or more pharmaceutically acceptable carriers; wherein the first and second compositions collectively comprise a twelve carbon chain fatty acid or twelve carbon chain acylglycerol, an omega 3 fatty acid, one or more vitamins, nutrients or minerals, and optionally one or more pharmaceutically acceptable carriers.

In a specific embodiments of the kits of the present invention, the one or more vitamins, nutrients or minerals from the first composition may be selected from one or more of the group consisting of vitamin D, iodine, vitamin B9, vitamin B12, and iron; and the one or more vitamins, nutrients or minerals from the second composition are selected from one or more of the group consisting of vitamin D, iodine, vitamin B9, vitamin B12, and iron. In a specific embodiment, vitamin D, if present, may be in an amount of about 200 I.U. to about 1600 I.U., iodine, if present, may be in an amount of about 100 μg to about 300 μg, vitamin B9, if present, may be in an amount of about 0.2 mg to about 1.5 mg, vitamin B12, if present, may be in an amount of about 2 μg to about 18 μg, and iron, if present, may be in an amount of about 13.5 mg to about 40.5 mg.

In a specific embodiment, the one or more vitamins, nutrients or minerals from the first composition may be selected from one or more of the group consisting of vitamin D, iodine, vitamin B1, vitamin B2, vitamin B3, vitamin B6, vitamin B9, vitamin B12, vitamin E, vitamin A, vitamin C, iron, zinc, copper, magnesium, and omega 3 fatty acids. In another embodiment, the vitamins, nutrients or minerals may be selected from one or more of the group consisting of vitamin D, iodine, vitamin B1, vitamin B2, vitamin B3, vitamin B6, vitamin B7, vitamin B9, vitamin B12, vitamin E, vitamin A, vitamin C, iron, zinc, copper, magnesium, omega 3 fatty acids, calcium, molybdenum, chlorine, vitamin K, manganese, selenium, choline, phosphorous, bioflavonoids, CoQ₁₀, chromium, alpha lipoic acid, choline, lutein and antioxidants.

In a specific embodiment, the first composition may be provided in a dosage form selected from the group consisting of a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution; and wherein the second composition may be provided in a dosage form selected from the group consisting of a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution. In another specific embodiment, the first composition and/or the second composition may be in the dosage form of a gelcap.

In another specific embodiment, the omega 3 fatty acids may comprise DHA. In a specific embodiment, the source of the DHA may be selected from one or more of the group consisting of algae oil and fish oil. In another embodiment, the DHA may be collectively present in an amount of about 100 mg to about 300 mg.

In another embodiment of the kits of the present invention, the twelve carbon chain fatty acid or twelve carbon chain acylglycerols may comprise one or more from the group consisting of lauric acid and monolaurin. In another embodiment, the source of lauric acid and/or monolaurin may be selected from one or more of the group consisting of algae oil and coconut oil. In a specific embodiment, the first composition and the second composition may collectively comprise lauric acid and/or monolaurin in an amount of about 30 mg to about 10 g. In a specific embodiment, the first composition and the second composition may collectively comprise lauric acid and monolaurin in an amount of about 30 mg to about 280 mg. In a specific embodiment, the first composition and the second composition may collectively comprise lauric acid in an amount of about 60 mg.

The present invention includes methods of administering compositions and kits for both prophylactic and therapeutic nutritional supplementation, and specifically, may include compositions and kits with twelve carbon chain fatty acid and/or twelve carbon chain acylglycerol. In another embodiment, the patient is in need of a carbon chain fatty acid or twelve carbon chain acylglycerol as an antiviral, a bactericidal, an antifungal or one or more combinations thereof. In another specific embodiment, the patient is human. In another embodiment, the human is a woman. In another embodiment, the woman is pregnant, prenatal or breast feeding.

In another embodiment, the kits of the present invention may comprise two to seven compositions, wherein the two to seven compositions collectively comprise a twelve carbon chain fatty acid or twelve carbon chain acylglycerol, one or more vitamins, nutrients or minerals; and one or more inactive ingredients. In a specific embodiment, the twelve carbon chain fatty acid or twelve carbon chain acylglycerol may be in the form selected from one or more of the group consisting of lauric acid and monolauirn and collectively may be present in an amount of about 30 mg to about 10 g. In another specific embodiment, the methods may include administering the kits comprising two to seven compositions to a patient.

### DETAILED DESCRIPTION OF THE INVENTION

It is understood that the present invention is not limited to the particular methodologies, protocols, fillers, and excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, kits and materials are described, although any methods, kits, and materials similar or equivalent to those described herein can be used in the practice or testing of the present invention. All references cited herein are incorporated by reference herein in their entirety.

The term “disease state” as used herein, may comprise any state in which one or more organs or components of an organism malfunction. The term “disease state” may refer to any deterioration of any component of a patient's body and specifically a human patient's body. The term “disease state” may refer to any deficiency of any compound necessary for the maintenance or function of any component of any organism. The term “disease state” may refer to any condition in which a body contains toxins, produced by microorganisms that infect the body or by body cells through faulty metabolism or absorbed from an external source. “Disease states” may be adverse states caused by any diet, any virus, fungi or any bacteria. “Disease states” may comprise disorders associated with pregnant females such as, for example, osteomalacia and preeclampsia and disorders associated with a fetus such as, for example, neural tube defects and various fetal abnormalities. “Disease states” may comprise any pulmonary disorder such as, for example, bronchitis, bronchiectasis, atelectasis, pneunomia, diseases caused by inorganic dusts, diseases caused by organic dusts, any pulmonary fibrosis, and pleurisy. “Disease states” may comprise any hematological/oncological disorder such as, for example, anemia, hemophilia, leukemia, and lymphoma. A “disease state” may comprise any cancer such as, for example, breast cancer, lung cancer, prostate cancer, pancreatic cancer, liver cancer, stomach cancer, testicular cancer, ovarian cancer, skin cancer, cancer of the brain, cancer of the mouth, cancer of the throat, and cancer of the neck. “Disease states” may comprise any disorder of the immune system such as, for example, acquired immune deficiency syndrome (AIDS), AIDS-related complex, infection by any strain of any human immunodeficiency virus (HIV), and other viruses or pathogens such as bacteria, fungi and parasites. A “disease state” may comprise any cardiovascular disorder such as, for example, arterial hypertension, orthostatic hypotension, arteriosclerosis, coronary artery disease, cardiomyopathy, any arrhythmia, any valvular heart disease, endocarditis, pericardial disease, any cardiac tumor, any aneurysm, and any peripheral vascular disorder. “Disease states” may comprise any hepatic/biliary disorder such as, for example, jaundice, hepatic steatosis, fibrosis, cirrhosis, hepatitis, any hepatic granuloma, any liver tumor, cholelithiasis, cholecystitis, and choledocholithiasis. A “disease state” may include a viral infection such as from HIV, herpes virus (HSV-1 and HSV-2), the virus that causes vesicular stomatitis (VSV), measles virus, herpes viridae, human lymprotropic virus, vesicular stomatitis virus, visna virus, cytomegalovirus, Epstein-Ban virus, influenza virus, pneumonovirus, Sarcoma virus, Syncitial virus and Rubeola virus. A “disease state” include a fungal infection such as from Candida albicans and Giardia lamblia. A “disease state” may include a bacterial infection such as from Staphylococcus, Corynebacerium, Bacillus, Listeria and Streptococcus bacteria, and include species such as Staphylococcus aureus, bacillus anthracis, Helicobacter pylori and, Listeria monocytogenes, and Streptococus agalactiae.

The term “patient,” as used herein, comprises any and all organisms and includes the term “subject.” “Patient” may refer to a human or any other animal. “Patient” may also refer to a fetus.

The phrase “co-administration” refers to administration of two or more compositions to a patient together, which includes administration at about the same time or within a certain specific or desired time.

The phrase “chewable form” refers to any relatively soft compositions that are chewed in the mouth after oral administration, may have a pleasant taste and mouthfeel, and may quickly break into smaller pieces and may begin to dissolve after chewing such that they can be swallowed substantially as a solution.

The phrase “dissolvable form” refers to any compositions that dissolve into a solution in the mouth. Such compositions, in one embodiment, may dissolve within about 60 seconds or less after placement in the mouth without any chewing.

The term “mouthfeel” refers to non-taste-related aspects of the pleasantness experienced by a person while chewing or swallowing a nutritional supplement. Aspects of mouthfeel include, for example and without limitation, the hardness and brittleness of a composition, whether the composition is chewy, gritty, oily, creamy, watery, sticky, easily dissolved, astringent, effervescent, and the like, and the size, shape, and form of the composition (tablet, powder, gel, etc.).

The phrase “pharmaceutically acceptable,” as used herein, refers to those compounds, materials, compositions and/or dosage forms which are, within the scope of sound pharmaceutical/medical judgment, suitable for use in contact with the tissues of human beings and animals without excessive toxicity, irritation, allergic response, or other problem or complication, commensurate with a reasonable benefit/risk ratio. Thus, the phrase “pharmaceutically acceptable carriers,” as used herein, refers to such suitable compounds and materials defined above that may be added to the dosage form to assist in satisfactory processing of the dosage form or provide desirable physical characteristics to the dosage form. For example, “pharmaceutically acceptable carriers” may include, but is not limited to, binders, diluents, lubricants, glidants, colorants, emulsifiers, disintegrants, starches, water, oils, alcohols, preservatives, and sugars. In another example, “pharmaceutically acceptable carriers” refers to dosage forms such as capsules, caplets, gel-caps used with, for example, the compositions of the present invention comprising or consisting of lauric acid. Thus, “pharmaceutically acceptable carriers” in gel-caps may be in for example, liquid or oil form, and may include a filler or other appropriate liquid vehicle and may be used with lauric acid and their equivalents.

The phrase “swallowable form” refers to any compositions that typically do not or are not configured to readily dissolve when placed in the mouth and may be swallowed whole, preferably without any, or with minimal, chewing or discomfort. Such compositions, in one embodiment, may have a shape containing no sharp edges and a smooth, uniform and substantially bubble free outer coating.

The term “dosage form,” as used herein, is the form in which the dose is to be administered to the subject or patient. The drug or supplement is generally administered as part of a formulation that includes nonmedical agents. The dosage form has unique physical and pharmaceutical characteristics. Dosage forms, for example, may be solid, liquid or gaseous. “Dosage forms,” may include for example, a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution. In a specific embodiment, the dosage form may be a gelcap.

The term “substantially free of added” as used herein, means free from therapeutically effective amounts of compounds when administered in suggested doses, but may include trace amounts of compounds in non-therapeutically effective amounts. For example, a composition of the present invention that included an inactive ingredient that is a salt or compound including a mineral would still be substantially free of added minerals. For example, trace amounts of titanium dioxide may be provided. Titanium dioxide which is an effective opacifer in powder form, where it is employed as a pigment to provide whiteness and opacity to numerous pharmaceutical products.

As used herein, the terms “inactive,” “inert,” “excipient,” and/or “formulatory” refer to any compound that is an inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

By “active ingredient,” then, is meant any compound intended to furnish pharmacological activity or other direct effect in the diagnosis, cure, mitigation, treatment and/or prevention of disease or a condition. See 21 C.F.R. 210.3(b)(7). Further, “active ingredients” include those compounds of the composition that may undergo chemical change during the manufacture of the composition and be present in the final composition in a modified form intended to furnish an activity or effect. Id. These include the vitamins, minerals, including twelve carbon chain fatty acids and twelve carbon chain acylglycerols of the compositions and kits of the present invention.

The term “administrable” defines a composition that is able to be given to a patient. Likewise, “administering” refers to the act of giving a composition to a patient or otherwise making such composition available to a patient or the patient taking a composition.

As used herein, the term “about,” when located before a dosage amount or dosage range of a specific ingredient, refers to an amount or range closely above and/or closely below the stated amount or range that does not manifestly alter the therapeutic effect of the specific ingredient from the stated amount or range and is meant to encompass at least all equivalents of that amount.

The compositions, kits and methods of the present invention provide supplementation of twelve carbon chain fatty acids and twelve carbon chain acylglycerols, compounds which may have antibacterial, antiviral, and antifungal properties and thus provide an overall anti-infectious benefit. The compositions, kits and methods of the present invention may be administered to or directed to a patient such as a human or any other organism. The compositions, kits and methods of the present invention provide supplementation of twelve carbon chain fatty acids such as lauric acid and/or twelve carbon chain acylglycerols such as monolaurin. The compositions, kits and methods of the invention may also include other twelve carbon chain fatty acids and/or twelve carbon chain acylglycerols, and specifically precursors or metabolites that provide these beneficial anti-infectious properties.

The compositions, kits and methods of the present invention may also provide these anti-infectious benefits of twelve carbon chain fatty acids and/or twelve carbon chain acylglycerols and also optimize good health by also utilizing vitamin, mineral and nutritional supplementation. The compositions, kits and methods of the present invention may also provide these anti-infectious benefits of twelve carbon chain fatty acids and/or twelve carbon chain acylglycerols, coupled with vitamin, mineral and nutritional supplementation during nutritionally volatile or physiologically stressful periods such as those including, by way of example and without limitation, pregnancy, lactation, or any disease state. Vitamin and mineral needs are almost universally increased throughout these periods. Increased needs during physiologically stressful states such as pregnancy, lactation or disease state may result from elevated metabolic demand, increased plasma volume, increased quantities of circulating red blood cells and decreased concentrations of nutrients.

Optimizing specific nutrients before, during, and after the physiological processes of pregnancy and lactation can have profound, positive, and comprehensive impacts on the overall wellness of the developing and newborn child as well as on the safety and health of the mother. Black, 85 (Supp.) BRIT. J. NUTR. S193-97 (2001); Scholl et al., 146 AMER. J. EPIDEM. 134-41 (1997). Indeed, a study reviewing the consumption of fatty acids demonstrates that these consumed compounds, among others, are quickly and significantly increased in the mother's milk. Francois et al., AM J CLIN NUTR, 67(2): 301-8 (1998). For example, fatty acids, such as lauric acid, increased significantly in human milk within 6 hrs of consumption by the lactating mother. Id. Further, a single meal of a particular fat may significantly affect the breast milk fatty acid composition for 1-3 days, and the maximum increase will probably occur during the first 24 hr. Id.

Further, nutrients provided to a mother reach the fetus. Specifically, it is established that substrates for growth and development, for example, circulate within the same pathways that carry drugs to and waste products from the fetus. Exchanges of material between mother and fetus occur primarily in the placenta, where villi containing fetal capillaries protrude into sinuses (intervillous spaces). Maternal arterial blood spurts into these spaces, then drains into maternal uterine veins to be returned to the maternal systemic circulation. Solutes in maternal blood cross the epithelial cells and connective tissue of the villi and the endothelium of the fetal capillaries; these solutes are then carried to the fetus by placental veins, which converge into the umbilical vein. THE MERCK MANUAL OF DIAGNOSIS AND THERAPY (Mark H. Beers, M. D. et al., 18th ed. 2006). For example, there is evidence that a diet that includes twelve carbon chain fatty acids or twelve carbon chain acylglycerols heavily influences the blood levels; thus these benefits may be provided to the fetus when provided in a pregnant women's diet. dela Paz et al., PHILLIPINE JOURNAL OF INTERNAL MEDICINE, The Effect of Virgin Coconut Oil on Lipid Profile and Fasting Blood Sugar: A Phase I Clinical Trial, 48(2); 1-7 (2010). Accordingly, the diet of a mother or pregnant women may have a substantial and important influence on the diet of the fetus or infant.

Twelve carbon chain fatty acids or twelve carbon chain acylglycerol supplementation may provide anti-infectious benefits to adult patients, and the fetus or infant due to the mother's diet, and may be specifically useful for the prevention or treatment of diseases caused by microbial infection. In a specific embodiment, the compositions, kits and methods of the present invention may be utilized or administered for the prevention or treatment or reduction of severity of infection of and by HIV, herpes virus (HSV-1 and HSV-2), the virus that causes vesicular stomatitis (VSV), measles virus, herpes viridae, human lymprotropic visusess, vesicular stomatitis virus, visna virus, cytomegalovirus, Epstein-Ban virus, influenza virus, pneumonovirus, Sarcoma virus, Syncitial virus and Rubeola virus. In another embodiment, the present invention may be utilized or administered for the prevention or treatment or reduction of severity of infection of and by bacteria. In a specific embodiment, the bacteria may be gram-positive bacteria. Indeed, there is evidence that twelve carbon acylglycerols such as monolaurin inhibit the effects of exotoxins from Gram-positive bacteria. Peterson et al., BIOCHEMISTRY, 45(7): 2387-97 (2006). By way of example, the bacteria may be Staphylococcus, Corynebacerium, Bacillus, Listeria and Streptococcus. In another specific embodiment, the species may be Staphylococcus aureus, bacillus anthracis, Helicobacter pylori, Listeria monocytogenes, and Streptococus agalactiae. Monolaurin has been studied, for example, for its bactericidal activity against Helicobacter pyori. Bergsson et al, INT J ANTIMICRO AGENTS, Bactericidal effects of fatty acids and monoglycerides on Helicobacter pylori. 20: 258 (2002). Monolaurin has also demonstrated activity against multiple fungi and viruses. Kristmundsdottir et al., J PHARM SCI, 88(10): 1011 (1999); Lieberman et al., ALTERNATIVE & COMPLEMENTARY THERAPIES, 310-314 (December, 2006). In a specific embodiment, the compositions, kits and methods of the present invention may be utilized or administered for the prevention or treatment or reduction in the severity of infection of fungal infections such as from Candida albicans and Giardia lamblia.

Accordingly, the compositions, kits and methods of the present invention may utilize or administer various twelve carbon chain fatty acids, many of which have multiple health benefits. In one example, lauric acid, a saturated twelve carbon chain fatty acid, contains many antibacterial, antiviral, and/or antifungal properties and thus has an overall anti-infectious benefit. Id. In another example, Lauroleic acid, an unsaturated twelve carbon fatty acid, may also provide similar benefits, which has been described as a natural metabolite of lauric acid in rat hepatocytes and may be present in milk. Legrand et al., LIPIDS, 37:569 (2002). Acylglycerols, which contain one to three fatty acid chains covalently bonded to a glycerol by an ester bond, can be precursors or metabolites of fatty acids likewise may also provide multiple health benefits and antimicrobial properties. Monolaurin, for example, is a twelve carbon acylglycerol of the fatty acid lauric acid and is a metabolite of lauric acid. Monolaurin possesses such antibacterial, antiviral, and antifungal properties, with greater activity than lauric acid. Lieberman et al., ALTERNATIVE & COMPLEMENTARY THERAPIES, 310-314 (December, 2006). In one example, the antibacterial activity of medium chain fatty acids and their acylglycerols were studied for antibacterial activity. Batovska et al., POL J MICROBIOL. Antibacterial study of the medium chain fatty acids and their 1-monoglycerides: individual effects and synergistic relationships, 58(1): 43-47 (2009). The study determined that that the monoacylglycerols were more active than their fatty acids, with monolaurin being the most active. Id.

The supplementation of twelve carbon chain fatty acids and/or twelve carbon chain acylglycerols may be beneficial to more than just the specific patient consuming these compounds. A diet that includes twelve carbon chain fatty acids or twelve carbon chain acylglycerols such as lauric acid and monolaurin translates into more of these compounds in mothers milk and thus may provide the same overall anti-infectious benefits to an infant. Francois et al., AM J CLIN NUTR, 67(2): 301-8 (1998). Indeed, numerous fatty acids, such as lauric acid, increased significantly in human milk within 6 hrs of consumption by the mother. Id. This study thus provides important information about the acute effect of the maternal diet on the availability of fatty acids for the breast-feeding infant. A single meal of a particular fat may significantly affect the breast milk fatty acid composition for 1-3 days, and the maximum increase will probably occur during the first 24 hr. Id. Even further, there is evidence that a diet of twelve carbon chain fatty acids or twelve carbon chain acylglycerols heavily influences the blood levels; thus these benefits may be provided to the fetus when provided in a pregnant women's diet. dela Paz et al., PHILLIPINE JOURNAL OF INTERNAL MEDICINE, The Effect of Virgin Coconut Oil on Lipid Profile and Fasting Blood Sugar: A Phase I Clinical Trial, 48(2):1-7 (2010). Accordingly, the diet of a mother or pregnant women may have a substantial and important influence on the fetus or infant for obtaining the multiple anti-infectious benefits of twelve carbon chain fatty acids or twelve carbon chain acylglycerols in the compositions and methods of the present invention.

In a specific embodiment of the present invention, the twelve carbon chain fatty acid or twelve carbon chain acylglycerol may be in the form of lauric acid, glycerol monolaurate (monolaurin), dilaurin, trilaurin, a twelve carbon chain monoacylglycerol, a twelve carbon chain unsaturated fatty acid, a diacylglycerol comprising at least one twelve carbon chain and a triacylglyceral comprising at least one twelve carbon atom chain.

The twelve carbon chain fatty acid may be saturated or unsaturated. For example, a saturated twelve carbon chain fatty acid (i.e., lauric acid) has the following structure:

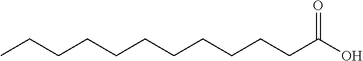

If the twelve carbon chain fatty acid is unsaturated, there may be one or more double bonds on the carbon chain.

Further, the one or more double bonds may be located between the two and the three carbon:

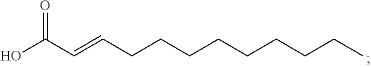

between the three and the four carbon:

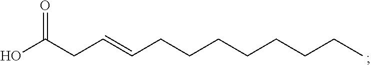

between the four and the five carbon:

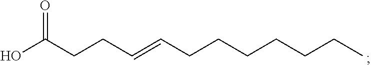

between the five and the six carbon:

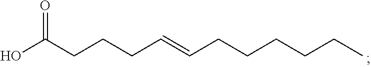

between the six and the seven carbon;

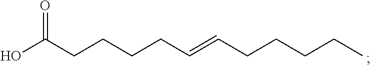

between the seven and the eight carbon:

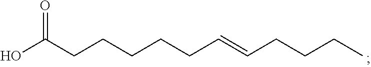

between the eight and the nine carbon:

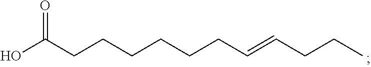

between the nine and the ten carbon:

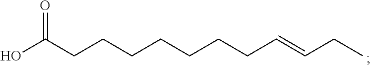

between the ten and the eleven carbon:

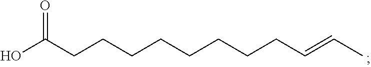

and between the eleven and twelve carbon:

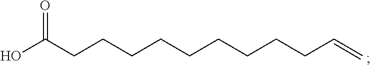

In another embodiment, the double bond carbons may be in either the cis or the trans configuration, or in the Z or the E configuration. The compounds provided directly above are all depicted in the trans or Z configuration. The unsaturated twelve carbon chain fatty acids may be in the cis or E configuration, for example:

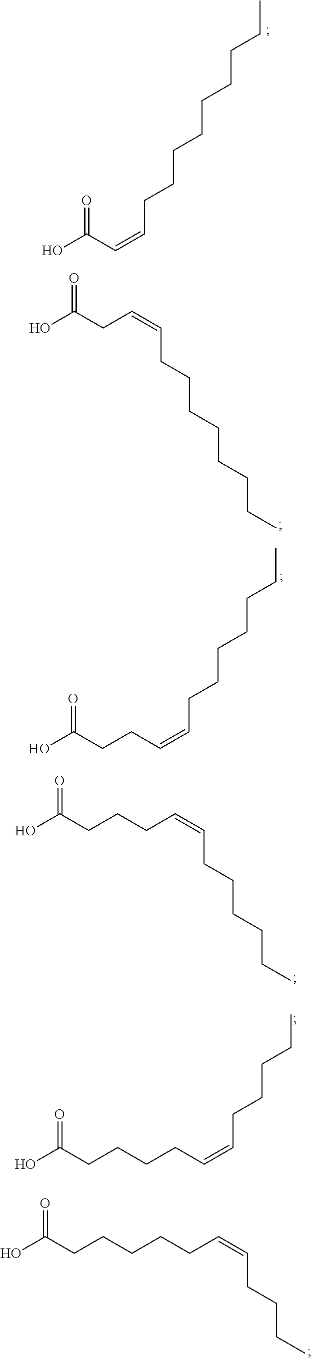

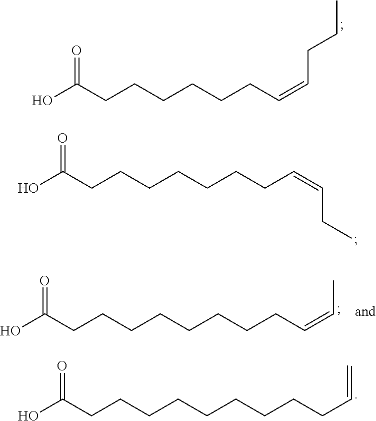

In another example, the twelve carbon chain fatty acid that is unsaturated may be linderic acid. Linderic acid has a double bond between the four and five carbon and has been found as the major fatty acid (47%) in various seed oils. Hopkins C Y et al., LIPIDS, 1:118 (1961). In another example, the twelve carbon chain fatty acid that is unsaturated may include lauroleic acid. Lauroleic acid has been described as a natural metabolite of lauric acid in rat hepatocytes, Legrand P et al., LIPIDS 37, 569 (2002), and may be present in milk. In another example, the twelve carbon chain fatty acid may be in any form of 2-dodecenoic acid, 3-dodecenoic acid, 4-dodecenoic acid, 5-dodecenoic acid, 6-dodecenoic acid, 7-dodecenoic acid, 8-dodecenoic acid, 9-dodecenoic acid, 10-dodecenoic acid, and 11-dodecenoic acid, and specifically, these compounds may be in the cis or trans configuration or the E or Z configuration.

In another embodiment of the present invention, the twelve carbon chain acylglycerol may be a twelve carbon chain monoacylglycerol, a diacylglycerol comprising at least one twelve carbon chain and a triacylglycerol comprising at least one twelve carbon atom chain. Possible combinations are depicted below in saturated forms:

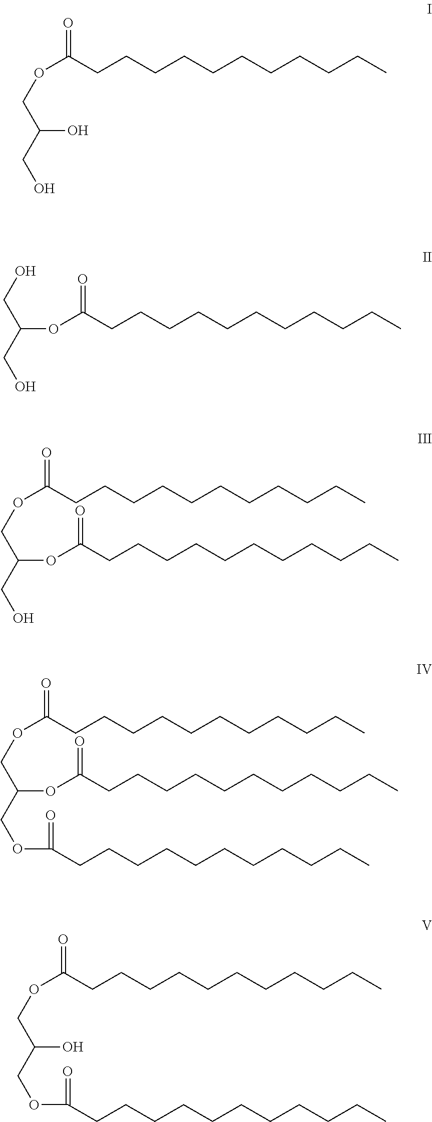 [SMILES: CCCCCCCCCCCC(=O)OCC(O)CO ; CCCCCCCCCCCC(=O)OC(CO)CO ; CCCCCCCCCCCC(=O)OCC(CO)OC(=O)CCCCCCCCCCC ; CCCCCCCCCCCC(=O)OCC(COC(=O)CCCCCCCCCCC)OC(=O)CCCCCCCCCCC ; CCCCCCCCCCCC(=O)OCC(O)COC(=O)CCCCCCCCCCC]

Accordingly, in the specific embodiments, the monoacylglycerol may include the esterified fatty acid on either the 1 carbon of glycerol (compound I) or the 2 carbon of glycerol (compound II). In another specific embodiment, the diacylglycerols may include the esterified fatty acid on the 1 and 2 carbon of glycerol (compound III) or on the 1 and 3 carbon of glycerol (compound V). In another specific embodiment, the triacylglycerol may include the esterified fatty acid on the 1, 2 and 3 carbon of glycerol (compound IV) In a specific embodiment, the monoacylglycerol may be glycerol monolaurate (monolaurin), dilaurin, and trilaurin.

In another embodiment, the acylglycerol may include fatty acids as described above that are esterified with glycerol. The esterified twelve carbon chain fatty acid may be saturated or unsaturated. If the esterified twelve carbon chain fatty acid is unsaturated, there may be one or more double bonds on the carbon chain. Further, the one or more double bonds may be located between the two and the three carbon, between the three and the four carbon, between the four and the five carbon, between the five and the six carbon, between the six and the seven carbon, between the seven and the eight carbon, between the eight and the nine carbon, between the nine and the ten carbon, between the ten and the eleven carbon, and between the eleven and twelve carbon. In another embodiment, the double bond carbons may be in either the cis or the trans configuration, or in the Z or the E configuration. For example, the esterified twelve carbon chain fatty acid that is unsaturated may be linderic acid. In another example, the esterified twelve carbon chain fatty acid that is unsaturated may include lauroleic acid. In another example, the esterified twelve carbon chain fatty acid may be 2-dodecenoic acid, 3-dodecenoic acid, 4-dodecenoic acid, 5-dodecenoic acid, 6-dodecenoic acid, 7-dodecenoic acid, 8-dodecenoic acid, 9-dodecenoic acid, 10-dodecenoic acid, and 11-dodecenoic acid. In a specific embodiment, these compounds may be in the cis or trans formation or the E or Z formation. In another specific embodiment, these fatty acids may be esterified on the 1, 2 or 3 carbon of glycerol.

In another specific embodiment of the present invention, the twelve carbon chain fatty acid or twelve carbon chain acylglycerol, such as lauric acid, is derived from a natural source, is synthesized or is genetically modified or engineered. In a specific embodiment, the twelve carbon chain fatty acid or twelve carbon chain acylglycerol may derived from a synthetic source. In a specific embodiment, the synthetic source is due to chemical modification of a compound to arrive at the twelve carbon chain fatty acid or twelve carbon chain acylglycerol. Indeed, acylglycerols are currently manufactured on a scale by for example, continuous chemical glycerosis of fats and oils. Langone et al., 2^{nd} Mercosur Congress on Chemical Engineering, 4^{th} Mercosure Congress on Prexx Systems Engineering, Solvent and Surfactant Free Synthesis of Monolaurin from Glycerol and Lauric acid, 2004. In one embodiment, acylglycerides may be synthesized by glycerolysis and direct esterification of glycerol with fatty acids. In one embodiment, monoglycerides and diglycerides may be prepared from the hydrolysis of triglycerides. In another embodiment, fatty acids may be synthesized, for example by hydrolysis of acylglycerides.

In a specific embodiment, the twelve carbon chain fatty acid or twelve carbon chain acylglycerol may derived from a natural source. Twelve carbon chain fatty acid or twelve carbon chain acylglycerols are known to be in a concentrated amount in various oils from, for example, palm trees and coconut oils. Indeed, lauric acid is the main fatty acid in coconut oil and in palm kernel oil. dela Paz et al., PHILLIPINE JOURNAL OF INTERNAL MEDICINE, The Effect of Virgin Coconut Oil on Lipid Profile and Fasting Blood Sugar: A Phase I Clinical Trial, 48(2): 1-7 (2010). In another embodiment, the natural source may be from an algae. Algae is known to be common source to produce fatty acids, such as DHA. See, for example, DHA in oil form, such as DHASCO®-T vegetable oil from micro-algae (Martek Biosciences Corporation, Columbia, Md.), or DHA from DHAgold®, life'sDHA™(DHASCO® and Martek Oil C53-0100. These oils contain a variety of other fatty acids and acylglycerols, such as lauric acid. Accordingly, in a specific embodiment, the natural source may be algae oil, coconut oil, babassu palm oil, and palm kernel oil.

In another embodiment, the source of twelve carbon chain fatty acid or twelve carbon chain acylglycerol is selected from one or more of the group consisting of algae oil, Martek algae oil (from Martek Biosciences Corporation, Columbia, Md.), coconut oil, babassu palm oil, palm kernel oil, ouricuri oil, tucum oil, muru-muru oil, jaboty kernel oil, khakan kernel oil, dika nut oil, ucuhuba butter, cinnamon oil, Brachyandra Calophylla seed oil, California laurel seed oil, camphor kernel fat, coconut oil, cohune nut oil (Palm Oil), Cuphea seed oil, Heteranthus epilobiifolia seed oil, Irvingia Gabonesis Kernel Fat (Dika Fat), jack bean oil, Khakan fat, Pelu fat, Laurel Berry (Bay Berry) oil, Lindera Umbellata seed oil, Ouricouri Tallow, palm kernel oil, Pindo Palm kernel oil, Pisa oil, Spicebush Kernel fat, Tucum (Aoiara) Kernel oil, cuphea plants, Lauraceae plants, Umbelliferae plants (Apiaceae plants), Arecaceae palms, Lythraceae plants, fruits of Laurus nobilis, qualea plants, Actinodaphne seed oil, cohune palms (Attalea cohune, rain tree, american oil palm, corozo palm or manaca palm), Pycanthus kombo (african nutmeg), Virola surinamensis (Wild nutmeg), peach palm seed, betel nut, date palm seed, macadamia nut, plum, Watermelon seed, Citrullus lanatus (egusi melon), and Pumpkin flower.

In one embodiment, the algae oil, may be from the species, Crypthecodinium cohnii oil, Schizochytrium oil, micro-algal oil (Ulkenia sp. SAM2179) and Schizochytrium linacinum strain SC-1. In one embodiment, the Cuphea plants may be from the species C. carthagenensis, C. lutea, C. wrightii and C. melanium. In one embodiment, the Arecaceae palms may be from the species Attalea colenda. In one embodiment, the Lythraceae plants may be from the genus Cuphea. In one embodiment, the Qualea plants may be from the species Qualea grandiflora (Vochysiaceae family). In another embodiment, the Actinodaphne seed oil may be from the species, Actinodaphne hookeri (Laureceae family). In another embodiment, the Babassu Palm Oil (Brazil) may be from the species, Attalea speciosa, Martius syn and Orbignya phalerata. In another embodiment, the Brachyandra Calophylla Seed Oil may be from the species Brachyandra calophylla. In another embodiment, California Laurel Seed Oil may be from the species Umbellularia californica (Laureceae family). In another embodiment, the Camphor kernel fat may be from the species Cinnamomum camphora (Laureceae family). In another embodiment, the Coconut oil may be from the species Cocos nucifera. In another embodiment, the Cuphea Seed Oil may be from species Cuphea wrightii and Heteranthus epilobiifolia. In another embodiment, the, Heteranthus epilobiifolia Seed Oil may be from the species Heteranthus epilobiifolia. In another embodiment, the Irvingia Gabonesis Kernel Fat (Dika Fat) may be from the species Irvingia gabonensis. In another embodiment, the Jack Bean Oil may be from the species Canavalia ensiformis. In another embodiment, the Khakan Fat or Pelu fat may be from the species Salvadora oleoides and S. persica. In another embodiment, the Laurel Berry (Bay Berry) Oil may be from the species Laurus Nobilis. In another embodiment, the Lindera Umbellata Seed Oil may be from the species Lindera umbellata. In another embodiment, the Ouricouri Tallow may be from the species Syagrus coronate and Orbignya cohune. In another embodiment, the Palm Kernel Oil may be from the species Elaeis guineensis, Buttia capitata, Alphanes acanthophylla and Elaeis oleifera. In another embodiment, the Pindo Palm Kernel Oil is from the species Anecastrum romanozoffianum. In another embodiment the Pisa Oil may be from the species Actinodaphne hookeri. In another embodiment, the Spicebush Kernel Fat may be from the species Lindera benzoin. In another embodiment, the Tucum (Aoiara) Kernel Oil may be from the species Astrocarpum spp. In a specific embodiment, the twelve carbon chain fatty acid and/or twelve carbon chain acylglycerol may be partially purified, or substantially purified by conventional chromatographic techniques known by one of ordinary skill in the art.

In another embodiment, the twelve carbon chain fatty acid or twelve carbon chain acylglycerol may be derived from a genetically modified or engineered source. In a specific embodiment, the twelve carbon chain fatty acid or twelve carbon chain acylglycerol may be engineered by introducing a gene donor for the twelve carbon chain fatty acid or twelve carbon chain acylglycerol production into a plant. In one embodiment, the plant may be easy to grow, or easily adapted to extract the twelve carbon chain fatty acid or twelve carbon chain acylglycerol. In a specific embodiment, genes in fatty acid and oil biosynthesis may be cloned. In specific embodiment, these genes may be cloned and introduced into rapeseed. In one embodiment, the twelve carbon chain fatty acid or twelve carbon chain acylglycerol is genetically engineered using rapeseed oil and introducing a gene donor involved in the biosynthesis of the twelve carbon chain fatty acid or twelve carbon chain acylglycerol production. In another embodiment, the California bay laurel tree Umbellularia californica may be a gene donor for the twelve carbon chain fatty acid or twelve carbon chain acylglycerol production. In a specific embodiment, the engineered product may be Laurical™. In one specific embodiment, lauric acid and/or monolaurin derived from a genetically modified or engineered source.

In a specific embodiment, the twelve carbon chain fatty acid or twelve carbon chain acylglycerol may be lauric acid and/or monolaurin. In another embodiment, lauric acid and/or monolaurin may be provided in the compositions of the present invention in appropriate amounts to be effective as and antibacterial, antiviral, and antifungal. Lieberman et al., ALTERNATIVE & COMPLEMENTARY THERAPIES, 310-314 (December 2006). In a specific embodiment, the lauric acid and/or monolaurin may be present in an amount greater than about 20 mg. In another embodiment, the lauric acid and/or monolaurin, may be present in an amount of about 30 mg to about 10 g. In another embodiment, the lauric acid and/or monolaurin may be present in an amount of about 30 mg to about 280 mg. In another embodiment, the lauric acid may be present in an amount of about 60 mg.

In another specific embodiment, the twelve carbon chain fatty acid or twelve carbon chain acylglycerol may be included in specific ranges or amounts for each specific form. When provided in their specific forms, the provided numerical range or amount includes the amounts of the specific form and/or compounds that are equivalent to the specific form. For example, the twelve carbon chain fatty acid or twelve carbon chain acylglycerol may be in the form lauric acid and may be included in the amount of about 60 mg. Accordingly, in this example, “lauric acid in the amount of about 60 mg” would include 60 mg of lauric acid and/or its equivalents and would, for example, include a product having 60 mg monolaurin instead of lauric acid.

The compositions, kits and methods of the present invention may also provide the means to optimize good health by utilizing vitamin, mineral, and nutritional supplementation. In a specific embodiment, the compositions, kits and methods of the present invention may utilize additional fatty acids such as omega-3 fatty acids. Omega-3 fatty acids play integral roles in physiological mechanisms that serve to prevent, treat and/or alleviate the occurrence or negative effects of some diseases and has shown multiple health-promoting properties in adults. For example, omega-3 fatty acids are linked to health benefits such as preventing the occurrence of cancer, preventing the occurrence of heart disease, and are helpful in brain health and immune function. Indeed, omega-3 fatty acids include essential fatty acids linked to numerous health benefits, such as docahexaenoic acid (or docosahexaenoic acid, DHA), eicosapentaenoic acid (EPA) and α-linolenic acid (ALA). In another specific embodiment, the compositions, kits and methods of the present invention may comprise or use docahexaenoic acid (or docosahexaenoic acid, DHA). In another specific embodiment, the kits and methods of the present invention may comprise or use eicosapentaenoic acid (EPA). In another specific embodiment, the compositions, kits and methods of the present invention may comprise or use α-linolenic acid (ALA).

The omega-3 fatty acid DHA, a major component of fish oil, has been shown to be of particular importance, especially during pregnancy or for lowering blood pressure. Indeed, studies show that DHA, but not EPA, reduce ambulatory blood pressure and heart rate in hyperlipidemic men. T A Mori et al., HYPERTENSION. 34:253-260 (1999). The results of this study thus provide that DHA is the principal fatty acid in fish and fish oils that is responsible for beneficial blood pressure and heart rate effects in humans. Id. Further, DHA is vital for optimal fetal and infant brain/cognitive development, as well as for normal brain function throughout life. F M Rioux, O. Hernell et al., ACTA PAEDIATR 95(2):137-144 (2006). The sleep patterns of infants born to mothers with higher plasma phospholipid DHA suggest greater central nerve system maturity. S R Cheruku, C J Lammi-Keefe et al., AM J CLIN NUTR 76:608-613 (2002). Such studies have indicated a correlation between maternal DHA intake and intelligence quotient in the child. The direct correlation between brain development and systemic DHA status is secondary to the fact that DHA is taken up by the brain in preference to other fatty acids. Adequate DHA levels in pregnancy have also been correlated with optimizing the length of gestation and decreasing the risk of neurodevelopmental psychopathology. These critical findings have prompted the National Institute of Health (NIH) to recommend that pregnant women consume at least 300 mg of omega-3 fatty acids daily during pregnancy. N. Neurenger et al., NUTR REV 44:285-294 (1986); G. Hornstra et al., AM J CLIN NUTR 71:285 S-291S (2000); I B Helland et al., PEDIATRICS 111:E39-E44 (2003); F. Facchinetti et al., EUR REV MED PHARMACOL SCI 9(1):41-48 (2005); R K McNamara et al., PROSTAGLANDINS LEUKOT ESSENT FATTY ACIDS (29 Aug. 2006).

DHA is also important for the development of the infant retina and improving the visual acuity of the infant. C A Francois, W E Connor et al., AM J CLIN NUTR 77:226-233 (2003). Preterm infants have a more rapid development of visual acuity if fed human milk or formula enriched with DHA, compared to standard formula. M H Jorgensen, K F Michaelsen et al., LIPIDS 31(1):99-105 (1996). An increase in visual acuity has also been observed to develop more rapidly in term infants breast-fed from mothers whose diets are supplemented with DHA. Id.

In addition to the aforementioned benefit of DHA to the developing child, this essential fatty acid has also shown multiple health-promoting properties in adults. These include anti-thrombotic, anti-inflammatory and anti-atherosclerotic activity, all of which reduce the risk of heart disease. M Laidlaw and B J Holub, AM J CLIN NUTR 77:37-42 (2003). Inverse relationships have also been found between systemic levels of omega-3 fatty acids such as DHA and incidence and severity of mood disorders and depression, including postpartum depression. Therefore, introduction of omega-3 fatty acids into the mother's diet during pregnancy has a benefit to both child and mother. F B Hu et al., JAMA 287(14):1815-1821 (2002); C. Von Schacky et al., ANN INTERN MED 130:554-562 (1999); G. Parker et al., AM J PSYCHIATRY 163(6):969-978 (2006); S J Otto et al., PROSTAGLANDINS LEUKOT ESSENT FATTY ACIDS 69(3):237-243 (2003).

Intake of omega-3 fatty acids such as DHA not only leads to their incorporation into cell membrane lipids (B A Stoll, BR J NUTR 87(3):193-198 (2002)), but also storage in adipose tissue and secretion in breast milk. C A Francois, W E Connor et al., AM J CLIN NUTR 77:226-233 (2003). Although the human body can derive a limited amount of DHA from another fatty acid known as alpha-linolenic acid, this process is inefficient for optimal needs. A rich dietary source of direct DHA is fish. Id. However, some lactating women are vegetarians, have limited access to fish or simply do not like fish. A further problem with encouraging increased fish intake in pregnancy is that most species contain methyl mercury (MeHg) in various amounts. MeHg is a potent neurotoxin that can increase the risk of retarded cognitive development. This concern prompted both the United States Environmental Protection Agency in 2004 and the United States Food and Drug Administration in 2001 to issue advisories recommending that pregnant women modify their fish consumption. These recommendations have resulted in a reduced intake of fish during pregnancy, thus helping to protect against fetal MeHg related harm. However, this has concurrently reduced maternal intake of DHA. In fact, a recent dietary study of over 100 pregnant or nursing women in the United States showed an astonishingly low intake of DHA on average (60-80 mg/day), and a dangerously low percentage (<2) consumed the aforementioned recommended intake of 300 mg/day of DHA as set forth by the NIH. J T Cohen et al., AM J PREV MED, 29:353-365 (2005); U.S. Department of Health and Human Services, U.S. Environmental Protection Agency, “What you need to know about mercury in fish and shellfish,” Report EPA-823-F-04-009 (March 2004); E. Oken et al., OBSTET GYNECOL 102:346-351 (2003).

DHA may be obtained in solid form, such as in a whole-cell microbial product, or in liquid form, such as in an oil. An example of DHA in oil form is DHASCO®-T vegetable oil from micro-algae (Martek Biosciences Corporation, Columbia, Md.). In a specific composition, the DHA is DHAgold®, life'sDHA™(DHASCO®), Martek Oil C53-0100, any Algae Oil, Krill Oil and/or vegetarian DHA.

In a specific embodiment of the present invention, the source of DHA is one or more from animal, fish, plants, algae, microorganism production or production by genetic engineering. In another specific embodiment, the source is fish oil.

In another embodiment, the compositions, kits and methods of the present invention may utilize DHA derived from the source, algae. DHA derived from algae, as opposed to being derived from fish oil, has numerous beneficial effects. First, the DHA from algae does not have the “fishy” smell that can come with DHA from fish oil. Indeed, high doses of DHA from fish oil may result in the patient having an unappealing after taste or a slight “fishy” body odor or “fishy” odor on the patient's breath. Second, DHA derived from algae can be more easily regulated to assure consistency and further remove the risk of added chemicals or other dangers. For example, DHA from algae would not have the risk of being tainted with mercury as opposed to DHA from fish oil. Thus, DHA from algae provides pregnant women and neonate with DHA without this risk and dangers of mercury. In a specific embodiment, the source of DHA may be from algae oil. In another specific embodiment, the source of algae oil may be microalgae Schizochytrium sp, microalgae Crypthecodinium cohnii, microalgae Ulkenia sp. SAM2179, microalgae Schizochytrium linacinum strain SC-1. In another specific embodiment the source of DHA may be Martek Oil C53-0100.

In another specific embodiment, omega-3 fatty acids may be included in amounts ranging from about 100 mg to about 300 mg. In another specific embodiment, omega-3 fatty acids may be present in amounts ranging from about 180 mg to about 220 mg. In another embodiment, omega-3 fatty acids may be present in an amount of about 200 mg.

In another specific embodiment, omega-3 fatty acids may be included in specific ranges or amounts for each specific form. When provided in their specific forms, the provided numerical range or amount includes the amounts of the specific form and/or compounds that are equivalent to the specific form. For example, omega-3 fatty acids may be in the form of DHA and may be included in the amount of about 200 mg. Accordingly, in this example, “DHA in the amount of about 200 mg” would include 200 mg of DHA and/or its equivalents and would, for example, include a product having 200 mg EPA instead of DHA.

In another specific embodiment, omega-3 fatty acids in the form of DHA may be present in amounts ranging from about 100 mg to about 300 mg. In another specific embodiment, omega-3 fatty acids in the form of DHA may be present in amounts ranging from about 180 mg to about 220 mg. In another embodiment, omega-3 fatty acids in the form of DHA may be present in an amount of about 200 mg. In a specific embodiment, the DHA in these amounts may be provided in fish oil and/or algae oil.

In another embodiment, the compositions, kits and methods of the present invention may include omega-3 fatty acids with twelve carbon chain fatty acids and twelve carbon chain acylglycerols. In a specific embodiment, the omega-3 fatty acid may be DHA.

Fish oil and/or algae oil can provide a good source of omega-3 fatty acids, such as DHA; other fatty acids and acylglycerols may also be present. For example, lauric acid and monolaurin may be present in fish oil and algae oil. The amounts of these compounds, however, are often minimal regarding providing the necessary nutritional or antimicrobial effects to a patient. Indeed, the concentration may be so low, that a very large volume of fish oil and algae oil—beyond a compliant amount for a patient—would be needed to provide the necessary benefits. Accordingly, a supplemental source for twelve carbon chain fatty acids and twelve carbon chain acylglycerols such as lauric acid and monolaurin may be included in addition to the DHA source. In one embodiment, the supplemental source may be the oils and species provided above. In a specific embodiment, the supplemental source may be coconut oil. Indeed, coconut oil contains about 50% of the twelve carbon chain fatty acid lauric acid of the total fatty acid composition. dela Paz et al., PHILLIPINE JOURNAL OF INTERNAL MEDICINE, The Effect of Virgin Coconut Oil on Lipid Profile and Fasting Blood Sugar: A Phase I Clinical Trial, 48(2); 1-7 (2010).

Accordingly, in a specific embodiment of the invention, the compositions, kits and methods of the present invention may include omega-3 fatty acids with twelve carbon chain fatty acids and twelve carbon chain acylglycerols wherein the twelve carbon chain fatty acids and twelve carbon chain acylglycerols from more than one source. In a specific embodiment, the twelve carbon chain fatty acids and twelve carbon chain acylglycerols, such as lauric acid and monolaurin, may be from a source selected from one or more of the group consisting of fish oil, algae oil and coconut oil. In a specific embodiment, the source may include both algae oil and coconut oil. In another specific embodiment, the source may include both fish oil and coconut oil. Specific ranges and amounts of the twelve carbon chain fatty acids and twelve carbon chain acylglycerols may be provided from each source. For example, in one embodiment, the twelve carbon chain fatty acids and twelve carbon chain acylglycerols such as lauric acid and monolaurin may be present in an amount of about 5 mg to about 30 mg from algae oil and about 25 mg to about 250 mg from coconut oil. In another embodiment, the twelve carbon chain fatty acids and twelve carbon chain acylglycerols such as lauric acid and monolaurin may be present in an amount of about 5 mg to about 25 mg from algae oil and about 25 mg to about 100 mg from coconut oil. In a specific embodiment, the twelve carbon chain fatty acids and twelve carbon chain acylglycerols such as lauric acid and monolaurin may be present in an amount of about 10 mg from algae oil and about 50 mg from coconut oil.

In a specific embodiment, the ranges and amounts of twelve carbon chain fatty acids and twelve carbon chain acylglycerols may be coupled with specific amounts of omega 3 fatty acids. In a specific embodiment, the twelve carbon chain fatty acids and twelve carbon chain acylglycerols, such as lauric acid and monolaurin may be present in an amount of about 5 mg to about 30 mg from algae oil and about 25 mg to about 250 mg from coconut oil and omega 3 fatty acids, such as DHA may be present in amount of about 100 mg to about 300 mg. In a specific embodiment, the source of DHA may be provided from algae oil. In another embodiment, the twelve carbon chain fatty acids and twelve carbon chain acylglycerols, such as lauric and monolaurin, may be present in an amount of about 10 mg from algae oil and 50 mg from coconut oil and omega 3 fatty acids, such as DHA may present in amount of about 200 mg. In a specific embodiment, the source of DHA may be provided from algae oil. To accommodate for such amounts of both twelve carbon chain fatty acids and twelve carbon chain acylglycerols, such as lauric acid and monolaurin, and omega 3-fatty acids such as DHA in the compositions, kits and methods of the present invention, various ranges of these sources may be needed. For example, in one embodiment, coconut oil may be provided in an amount of about 50 mg to about 500 mg. In another embodiment, algae oil may be provided in an amount of about 200 mg to about 600 mg. In another embodiment, fish oil may be provided in an amount of about 200 mg to about 600 mg.

Proper nutrition is also important for maintaining health and preventing or reducing the severity of multiple diseases. Accordingly, the compositions, kits and methods of the present invention may also provide the means to optimize good health by utilizing other vitamins, minerals and nutrients. In a specific embodiment, the compositions, kits and methods of the present invention may comprise or use vitamins nutrients or minerals selected from one or more of the group consisting of vitamin D, iodine, vitamin B1, vitamin B2, vitamin B3, vitamin B6, vitamin B7, vitamin B9, vitamin B12, vitamin E, vitamin A, vitamin C, iron, zinc, copper, magnesium, calcium, molybdenum, chlorine, vitamin K, omega 3 fatty acids, manganese, selenium, choline, phosphorous, bioflavonoids, CoQ₁₀, chromium, alpha lipoic acid, choline, lutein and antioxidants. In a specific embodiment, specific forms or each vitamin, nutrient and mineral may be provided.

In a specific embodiment, the compositions, kits and methods of the present invention may comprise or use vitamin A. In a specific embodiment, vitamin A may be in the forms of retinol acetate (also known as retinyl acetate or vitamin A acetate), retinol (vitamin A alcohol), retinol palmitate (also known as retinyl palmitate or vitamin A palmitate), retinoic acid (tretinoin), retinal, beta-cryptoxanthin, alpha-carotene, beta-carotene, gamma-carotene, and provitamin A carotenoids.

In a specific embodiment of the compositions, kits and methods of the present invention, vitamin A may be included in amounts ranging from about 550 IU to about 1650 IU. In another specific embodiment, vitamin A may be included in amounts ranging from about 880 IU to about 1320 IU. In another specific embodiment, vitamin A may be included in amounts ranging from about 990 IU to about 1210 IU. In another embodiment, vitamin A may be included in an amount of about 1100 IU.

In another specific embodiment, vitamin A may be included in specific ranges or amounts for each specific form. When provided in their specific forms, the provided numerical range or amount includes the amounts of the specific form and/or compounds that are equivalent to the specific form. For example, vitamin A may be in the form of beta-carotene and may be included in the amount of about 1100 IU. Accordingly, in this example, “beta-carotene in the amount of about 1100 IU” would include 1100 IU of beta-carotene and/or its equivalents and would, for example, include a product having 1100 IU vitamin A palmitate instead of beta-carotene. The compositions, kits and methods of the present invention may comprise or use B-complex vitamins. In one embodiment, the B-complex vitamins that may be included in the compositions, kits and methods of the present invention comprise one or more of vitamin B1, vitamin B2, vitamin B3, vitamin B6, vitamin B9 and vitamin B12. In another embodiment, vitamin B7 may be included.

The compositions, kits and methods of the present invention may comprise or use vitamin B1. In another specific embodiment, vitamin B1 may be in the forms of thiamine, thiamine monophosphate, thiamine diphosphate, thiamine triphosphate, acetiamine, allithiamine, prosultiamine and S-acyl derivatives of thiamine such as benfotiamine, fursultiamine and salts and esters thereof.

In another specific embodiment, vitamin B1 may be included in amounts ranging from about 0.8 mg to about 2.4 mg. In another specific embodiment, vitamin B1 may be included in amounts ranging from about 1.3 mg to about 1.9 mg. In another specific embodiment, vitamin B1 may be included in amounts ranging from about 1.4 mg to about 1.75 mg. In another embodiment, vitamin B1 may be included in an amount of about 1.6 mg.

In another specific embodiment, vitamin B1 may be included in specific ranges or amounts for each specific form. When provided in their specific forms, the provided numerical range or amount includes the amounts of the specific form and/or compounds that are equivalent to the specific form. For example, vitamin B1 may be in the form of thiamine mononitrate and may be included in the amount of about 1.6 mg. Accordingly, in this example, “thiamine mononitrate in the amount of about 1.6 mg” would include 1.6 mg of thiamine mononitrate and/or its equivalents and would, for example, include a product having 1.6 mg allithiamine instead of thiamine mononitrate.

The compositions, kits and methods of the present invention may comprise or use vitamin B2. In a specific embodiment, vitamin B2 may be in the forms of flavin mononucleotide (FMN), flavin adenine dinucleotide (FAD), riboflavin (also known as 7,8-dimethyl-10-((2R,3R,4S)-2,3,4,5-tetrahydroxypentyl)benzo[g]pteridine-2,4 (3H,10H)-dione or lactoflavin) and riboflavin derivatives such as riboflavin-5′-monophosphate, riboflavin-5′-monobutyrate and riboflavin-5′-monopalmitate. In a specific embodiment of the present invention, vitamin B2 may be included in the form of riboflavin.

In another specific embodiment, vitamin B2 may be included in amounts ranging from about 0.9 mg to about 2.7 mg. In another specific embodiment, vitamin B2 may be included in amounts ranging from about 1.5 mg to about 2.2 mg. In another specific embodiment, vitamin B2 may be included in amounts ranging from about 1.6 mg to about 2 mg. In another embodiment, vitamin B2 may be included in an amount of about 1.8 mg.

In another specific embodiment, vitamin B2 may be included in specific ranges or amounts for each specific form. When provided in their specific forms, the provided numerical range or amount includes the amounts of the specific form and/or compounds that are equivalent to the specific form. For example, vitamin B2 may be in the form of riboflavin and may be included in the amount of about 1.8 mg. Accordingly, in this example, “riboflavin in the amount of about 1.8 mg” would include 1.8 mg of riboflavin and/or its equivalents and would, for example, include a product having 1.8 mg flavin mononucleotide instead of riboflavin.

The compositions, kits and methods of the present invention may comprise or use vitamin B3. In a specific embodiment, vitamin B3 may in the forms of niacin (nicotinic acid or pyridine-3-carboxylic acid), and nicotinamide (niacinamide) and salts and esters thereof. In a specific embodiment of the present invention, vitamin B3 may be included in the form of nicotinamide. In another specific embodiment, the present invention may include an equivalent molar amount of niacin.

In another specific embodiment, vitamin B3 may be included in amounts ranging from about 7.5 mg to about 22.5 mg. In another specific embodiment, vitamin B3 may be included in amounts ranging from about 12 mg to about 18 mg. In another specific embodiment, vitamin B3 may be included in amounts ranging from about 13.5 mg to about 16.5 mg. In another embodiment, vitamin B3 may be included in an amount of about 15 mg.

In another specific embodiment, vitamin B3 may be included in specific ranges or amounts for each specific form. When provided in their specific forms, the provided numerical range or amount includes the amounts of the specific form and/or compounds that are equivalent to the specific form. For example, vitamin B3 may be in the form of nicotinamide and may be included in the amount of about 15 mg. Accordingly, in this example, “nicotinamide in the amount of about 15 mg” would include 15 mg of nicotinamide and/or its equivalents and would, for example, include a product having 15 mg niacin instead of nicotinamide.

The compositions, kits and methods of the present invention may comprise or use vitamin B6. In a specific embodiment of the present invention, vitamin B6 may be included in the forms of pyridoxine, 3-hydroxy-4,5-bis(hydroxymethyl)-2-methylpyridine, 5′-deoxypyridoxal, 2-demethylpyridoxal (known as 2-norpyridoxal), 2-propyl-2-norpyridoxal, 2′-ethylpyridoxal, 6-methylpyridoxal, 2′-hydroxypyridoxal, 2-hydroxymethyl-2-demethylpyridoxal, 2-hydroxymethyl-2-norpyridoxal, 4′-deoxypyridoxine5′-phosphate, 5′-methylpyridoxal-5′-phosphate, pyridoxal N-oxide5′-phosphate, Pyridoxal, Pyridoxamine, Pyridoxine-5′-phosphate (PNP), pyridoxal-5′-phosphate (PLP) and pyridoxamine-5′-phosphate (PMP), and their salts and chelates thereof.

In another specific embodiment, vitamin B6 may be included in amounts ranging from about 1.2 mg to about 3.8 mg. In another specific embodiment, vitamin B6 may be included in amounts ranging from about 2.0 mg to about 3.0 mg. In another specific embodiment, vitamin B6 may be included in amounts ranging from about 2.25 mg to about 2.75 mg. In another embodiment, vitamin B6 may be included in an amount of about 2.5 mg.

In another specific embodiment, vitamin B6 may be included in specific ranges or amounts for each specific form. When provided in their specific forms, the provided numerical range or amount includes the amounts of the specific form and/or compounds that are equivalent to the specific form. For example, vitamin B6 may be in the form of pyridoxine hydrochloride and may be included in the amount of about 2.5 mg. Accordingly, in this example, “pyridoxine hydrochloride in the amount of about 2.5 mg” would include 2.5 mg of pyridoxine hydrochloride and/or its equivalents and would, for example, include a product having 2.5 mg pyridoxamine instead of pyridoxine hydrochloride.

The compositions, kits and methods of the present invention may comprise or use vitamin B9. Vitamin B9 is a generic name of a B-vitamin that includes multiple compounds with a general structure. For example, vitamin B9 encompasses the term folate, which itself is the generic name for many different forms of this water-soluble vitamin (vitamin B9), which is essential for DNA synthesis and, hence, cell division. Simpson et al., THE JOURNAL OF MATERNAL-FETAL AND NEONATAL MEDICINE, Micronutrients and women of reproductive potential: required dietary intake and consequences of dietary deficiency or excess. Part I—Folate, Vitamin B12, Vitamin B6, Epub 1-21, (2010). Indeed, folate encompasess numerous compounds that for example, are based on a pteridine ring, an aminobenzoic acid and one or more glutamic acid residues. Id. Folic acid (pteroglutamic acid or PGA) is a synthetic form of folate, and the first folate synthesised and used as a supplement. Id. The term folates may also be used in the generic sense to designate any members of the family of pteroylglutamates, or mixtures of them, having various levels of reduction of the pteridine ring, one-carbon substitutions and numbers of glutamate residues. PURE & APPL. CHEM., IUPAC-IUB Commission on Biochemical Nomenclature (CBN). Nomenclature and Symbols for Folic Acid and Related Compounds. Arch 59, No. 6: 833-836 (1987).

Vitamin B9, however, is not only defined by its structure, but also by its various functions. Indeed, vitamin B9 is essential for DNA synthesis and, hence, cell division and is required metabolically as a coenzyme in one-carbon transfer reactions. Simpson, supra. This vitamin has demonstrated the ability to prevent neural tube defects such as spina bifida caused by disturbed homocysteine metabolism. Vanderput et al., EXP. BIOL. MED. 243-70 (2001); DeFalco et al., 27 CLIN. EXP. OBSTET. GYNECOL. 188-90 (2000); Eskes, 27 CLIN. EXP. OBSTET. GYNECOL. 157-67 (2000); Locksmith & Duff, supra. Folic acid, a commonly used term synonymous with vitamin B9, is known to reduce the risk of multiple diseases. Clinical trials definitively demonstrated the effectiveness of folic acid supplementation in reducing the number of neural tube defects. Simpson et al., THE JOURNAL OF MATERNAL-FETAL AND NEONATAL MEDICINE, Micronutrients and women of reproductive potential: required dietary intake and consequences of dietary deficiency or excess. Part I—Folate, Vitamin B12, Vitamin B6, Epub 1-21, (2010). Indeed, folic acid supplementation in reducing the risk of neural tube defects and other congenital malformations is generally accepted. Pietrzik et al., CLIN PHARMACOKINET 49 (8): 535-548 (2010). Furthermore, evidence is accumulating to support a possible role of folic acid in the reduction in risk of other diseases, including dementia and certain types of cancer. Id. Lastly, folate or folate derivative thereof that increase blood folate levels, thereby reducing homocysteine levels, which is a common way to measure vitamin B9 effectiveness. Id.

Thus, in a specific embodiment of the present invention, vitamin B9 may include numerous forms. In a specific embodiment, vitamin B9 may be included in the form of folic acid. In another embodiment, vitamin B9 may be included one or more of the forms of folic acid, folacin, metafolin, folate and/or one or more natural isomers of folate including (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof and the salts and esters thereof. In another embodiment, vitamin B9 may be in the form of a folate or folate derivative thereof that is eventually converted to 5-methyl-tetrahydrofolic acid in the body and/or is absorbed into the bloodstream as 5-methyl-tetrahydrofolic acid. Folates, such as folic acid and folate, are eventually absorbed in the body and converted to L-5-methyl-tetrahydrofolic acid. In another embodiment, vitamin B9 may be in the form of a folate or folate derivative thereof that increases blood folate levels, thereby reducing homocysteine levels.

In another embodiment, vitamin B9 may be in the form of folate or reduced folates with various salts. In a specific embodiment, the folate and reduced folate are selected from the group consisting of D-glucosamine-folate, D-galactosamine-folate, D-glucosamine (6R,S)-tetrahydrofolate, D-glucosamine (6S)-tetrahydrofolate, D-glucosamine (6R)-tetrahydrofolate; D-galactosamine (6R,S)-tetrahydrofolate, D-galactosamine (6S)-tetrahydrofolate, D-galactosamine (6R)-tetrahydrofolate; D-glucosamine 5-methyl-(6R,S)-tetrahydrofolate, D-glucosamine 5-methyl-(6S)-tetrahydrofolate, D-glucosamine 5-methyl-(6R)-tetrahydrofolate; D-galactosamine 5-methyl-(6R,S)-tetrahydrofolate, D-galactosamine 5-methyl-(6S)-tetrahydrofolate, and D-galactosamine 5-methyl-(6R)-tetrahydrofolate.

In another specific embodiment, vitamin B9 may be included in amounts ranging from about 0.2 mg to about 1.5 mg. In another specific embodiment, vitamin B9 may be included in amounts ranging from about 0.8 mg to about 1.2 mg. In another specific embodiment, vitamin B9 may be included in amounts ranging from about 0.9 mg to about 1.1 mg. In another embodiment, vitamin B9 may be included in an amount of about 1.0 mg.

In another specific embodiment, vitamin B9 may be included in specific ranges or amounts for each specific form. When provided in their specific forms, the provided numerical range or amount includes the amounts of the specific form and/or compounds that are equivalent to the specific form. For example, vitamin B9 may be in the form folic acid and may be included in the amount of about 1.0 mg. Accordingly, in this example, “folic acid in the amount of about 1.0 mg” would include 1.0 mg of folic acid and/or its equivalents and would, for example, include a product having 1.0 mg 5-methyl-(6S)-tetrahydrofolic acid instead of folic acid.

The compositions, kits and methods of the present invention may comprise or use vitamin B12. In a specific embodiment of the present invention, vitamin B12 may be in one or more of the forms of cobalamin, methylcobalamin, 5′-deoxyadenosylcobalamin (adenosylcobalamin or cobamamide), cyanocobalamin, hydroxycobalamin and mecobalamin.

In another specific embodiment, vitamin B12 may be included in amounts ranging from about 2 μg to about 18 μg. In another specific embodiment, vitamin B12 may be included in amounts ranging from about 9.6 μg to about 14.4 μg. In another specific embodiment, vitamin B12 may be included in amounts ranging from about 10.8 μg to about 13.2 μg. In another embodiment, vitamin B12 may be included in an amount of about 12 μg.

In another specific embodiment, vitamin B12 may be included in specific ranges or amounts for each specific form. When provided in their specific forms, the provided numerical range or amount includes the amounts of the specific form and/or compounds that are equivalent to the specific form. For example, vitamin B12 may be in the form cyanocobalamin and may be included in the amount of about 12 μg. Accordingly, in this example, “cyanocobalamin in the amount of about 12 μg” would include 12 μg of cyanocobalamin and/or its equivalents and would, for example, include a product having 12 μg methylcobalamin instead of cyanocobalamin.

The compositions, kits and methods of the present invention may comprise or use vitamin B7. In a specific embodiment, vitamin B7 may be biotin.

In a specific embodiment of the present invention, vitamin B7 may be included in amounts ranging from about 150 μg to about 450 μg. In another specific embodiment of the present invention, vitamin B7 may be included in amounts ranging from about 240 μg to about 360 μg. In a further specific embodiment of the present invention, vitamin B7 may be included in amounts ranging from about 270 μg to about 330 μg. In another embodiment, vitamin B7 may be included in an amount of about 300 μg.

The compositions, kits and methods of the present invention may comprise or use vitamin C. In a specific embodiment of the present invention, vitamin C may be included in the forms of ascorbic acid, ascorbates (calcium or sodium ascorbate), dehydroascorbic acid and salts, ascorbyl palmitate, ascorbyl phosphates and salts (such as sodium or magnesium ascorbyl phosphate), ascorbyl tetraisopalmitate, tetrahexyldecyl ascorbate, ascorbyl sulfates and salts, acylated ascorbic acid derivatives (such as 6-O-acyl-2-O-alpha-D-glucopyranosyl-L-ascorbic acids), 6-bromo-6-deoxy-L-ascorbic acid, and ascorbate salts.

In another specific embodiment, vitamin C may be included in amounts ranging from about 8 mg to about 45 mg. In another specific embodiment, vitamin C may be included in amounts ranging from about 24 mg to about 36 mg. In another specific embodiment, vitamin C may be included in amounts ranging from about 27 mg to about 33 mg. In another embodiment, vitamin C may be included in an amount of about 30 mg.

In another specific embodiment, vitamin C may be included in specific ranges or amounts for each specific form. When provided in their specific forms, the provided numerical range or amount includes the amounts of the specific form and/or compounds that are equivalent to the specific form. For example, vitamin C may be in the form ascorbic acid and may be included in the amount of about 12 mg. Accordingly, in this example, “ascorbic acid in the amount of about 12 mg” would include 12 mg of ascorbic acid and/or its equivalents and would, for example, include a product having 12 mg ascorbyl palmitate instead of ascorbic acid.

The compositions, kits and methods of the present invention may comprise or use vitamin D. In a specific embodiment of the present invention, vitamin D may be in the forms of vitamin D3 (also known as calciol or cholecalciferol or colecalciferol), vitamin D2 (also known as calciferol, ergocalciol, ergocalciferol, ercalciol, Deltalin or Viosterol), previtamin D2, ergosterol, calcitriol (also known as 1,25-dihydroxycholecalciferol), 7-dehydrocholesterol, Vitamin D1, vitamin D4 (also known as 22-dihydroergocalciferol, 22,23-dihydroercalciol or (24S)-methylcalciol), vitamin D5 (also known as (24S)-Ethylcalciol or sitocalciferol), 7-dehydrositosterol, Lumisterol, 25-hydroxyvitamin D, all steroids that exhibit the biological activity of calciol, 25-fluorocalciol, (3S)-3-amino-3-deoxycalciol, 11-acetoxycalciol, calcidiol (also known as 25-hydroxycholecalciferol or calcifediol), ercalcitriol, calcitetrol, tacalciol (also known as tachysterol3), (5E)-isocalciol (also known as isovitamin D3), Dihydroercalciol (also known as dihydrotachysterol3), (1S)-Hydroxycalciol (also known as 1-hydroxycholecalciferol or alfacaleidol), (24R)-Hydroxycalcidiol (also known as 24(R),25-dihydroxycholecalciferol), Ercalcidiol, Ercalcitriol, Ertacalciol, (5E)-(10S)-10,19-Dihydroercalciol (also known as dihydrotachysterol2), (6Z)-Tacalciol (also known as precalciferol or pre-vitamin D), and (22E)-(24R)-Ethyl-22,23-didehydrocalciol also known as vitamin D6).

In one embodiment of the invention, vitamin D may be present in the amount ranging from about 200 IU to about 1600 IU. In another embodiment, vitamin D may be present in the amount ranging from about 750 IU to about 1250 IU. In another embodiment, vitamin D is present in the amount ranging form about 900 IU to about 1100 IU. In another embodiment, vitamin D is present in the amount of about 1000 IU.

In another specific embodiment, vitamin D may be included in specific ranges or amounts for each specific form. When provided in their specific forms, the provided numerical range or amount includes the amounts of the specific form and/or compounds that are equivalent to the specific form. For example, vitamin D may be in the form vitamin D3 and may be included in the amount of about 1000 IU. Accordingly, in this example, “vitamin D3 in the amount of about 30 mg” would include 1000 IU of vitamin D3 and/or its equivalents and would, for example, include a product having 1000 IU mg vitamin D2 instead of vitamin D3.

The compositions, kits and methods of the present invention may comprise or use vitamin E. In a specific embodiment of the present invention, vitamin E may be included in the forms of alpha, beta, gamma, and delta tocopherols in its natural or synthetic (dl) forms; alpha, beta, gamma, and delta tocotrienols in its natural or synthetic (dl) forms, dl-alpha tocopheryl derivatives such as dl-alpha tocopheryl esters, dl-alpha-tocopheryl acetate or succinate and d-alpha-tocopheryl acetate or dl-alpha tocopheryl phosphates (such as Ester-E®).

In another specific embodiment, vitamin E may be included in amounts ranging from about 5 IU to about 30 IU. In another specific embodiment, vitamin E may be included in amounts ranging from about 15 IU to about 25 IU. In another specific embodiment, vitamin E may be included in amounts ranging from about 18 IU to about 22 IU. In another embodiment, vitamin E may be included in an amount of about 20 IU.

In another specific embodiment, vitamin E may be included in specific ranges or amounts for each specific form. When provided in their specific forms, the provided numerical range or amount includes the amounts of the specific form and/or compounds that are equivalent to the specific form. For example, vitamin E may be in the form d-alpha-tocopheryl acetate and may be included in the amount of about 10 IU. Accordingly, in this example, “d-alpha-tocopheryl in the amount of about 10 IU” would include 10 IU of d-alpha-tocopheryl and/or its equivalents and would, for example, include a product having 10 IU alpha-tocotrienol instead of d-alpha-tocopheryl.

The compositions, kits and methods of the present invention may comprise or use iron. The, compositions, kits and methods of the present invention may include iron in the forms of elemental iron, in the form of a salt, chelated form, non-chelated form, chelated to an amino acid, carbonyl iron, ferrous gluconate, ferrous fumarate, polysaccharide iron complex, elemental polysaccharide iron, polysaccharide iron, ferrous (II)-bis-glycinate chelate, ferrous asparto glycinate, ferrous bisglycinate, ferrous bisglycinate hydrochloride, elemental ferrous bisglycinate, ferrous sulfate, ferronyl (micronized), as Iron Aid, iron protein succinylate, carbonyl iron, Sumalate iron, Heme iron complex, as Ferrochel amino acid chelate, Heme iron polypeptide as Proferrin-bovine source, as heme iron polypeptide (bovine source) as sodium iron EDTA (Ferrazone), ferric ammonium citrate, elemental iron, and ferric pyrophosphate.

In another specific embodiment, iron may be included in amounts ranging from about 13.5 mg to about 40.5 mg. In another specific embodiment, iron may be included in amounts ranging from about 21.6 mg to about 32.4 mg. In another specific embodiment, iron may be included in amounts ranging from about 24.3 mg to about 29.7 mg. In another embodiment, iron may be included in an amount of about 27 mg.

In another specific embodiment, iron may be included in specific ranges or amounts for each specific form. When provided in their specific forms, the provided numerical range or amount includes the amounts of the specific form and/or compounds that are equivalent to the specific form. For example, iron may be in the form polysaccharide iron complex and may be included in the amount of about 27 mg. Accordingly, in this example, “polysaccharide iron complex in the amount of about 27 mg” would include 27 mg of polysaccharide iron complex and/or its equivalents and would, for example, include a product having 27 mg ferrous fumarate instead of polysaccharide iron complex.

The compositions, kits and methods of the present invention may comprise or use iodine. In a specific embodiment, iodine may be in the forms of elemental iodine, iodized salt, Lugol's iodine, sodium iodide, potassium iodide, potassium iodate, nascent iodine, and Nano-Colloidal Detoxified Iodine.

In another specific embodiment, iodine may be present in the amounts ranging from about 100 μg to about 300 μg. In another embodiment, iodine may be present in the amounts ranging from about 160 μg to about 240 μg. In another embodiment, iodine may be present in the amounts ranging from about 180 μg to about 220 μg. In another embodiment, iodine may be present in the amount of about 200 μg.

In another specific embodiment, iodine may be included in specific ranges or amounts for each specific form. When provided in their specific forms, the provided numerical range or amount includes the amounts of the specific form and/or compounds that are equivalent to the specific form. For example, iodine may be in the form potassium iodide and may be included in the amount of about 200 μg. Accordingly, in this example, “potassium iodide in the amount of about 200 μg” would include 200 μg of potassium iodide and/or its equivalents and would, for example, include a product having 200 μg Nano-Colloidal Detoxified Iodine instead of potassium iodide.

The compositions, kits and methods of the present invention may comprise or use magnesium. In one specific embodiment of the present invention, magnesium may be included in the forms of elemental magnesium, in the form of a salt, in a chelated form, in a non-chelated form, magnesium acetate, magnesium carbonate, magnesium gluconate, magnesium chloride, magnesium citrate, magnesium silicate, magnesium stearate, magnesium sulfate, magnesium oxide, and magnesium chelated to an amino acid (magnesium glycinate, magnesium aspartate).

In another specific embodiment, magnesium may be present in the amounts ranging from about 2.5 mg to about 10 mg. In another embodiment, magnesium may be present in the amounts ranging from about 4 mg to about 6 mg. In another embodiment, magnesium may be present in the amounts ranging from about 4.5 mg to about 5.5 mg. In another embodiment, magnesium may be present in the amount of about 5 mg.

In another specific embodiment, magnesium may be included in specific ranges or amounts for each specific form. When provided in their specific forms, the provided numerical range or amount includes the amounts of the specific form and/or compounds that are equivalent to the specific form. For example, magnesium may be in the form magnesium oxide and may be included in the amount of about 5 mg. Accordingly, in this example, “magnesium oxide in the amount of about 5 mg” would include 5 mg of magnesium oxide and/or its equivalents and would, for example, include a product having 5 mg magnesium stearate instead of magnesium oxide.

The compositions, kits and methods of the present invention may comprise or use zinc. In a specific embodiment of the present invention, zinc may be provided in the forms of elemental zinc, in the form of a salt, in a chelated form, in a non-chelated form, zinc acetate, zinc gluconate, zinc picolinate, zinc sulfate and zinc oxide.

In another specific embodiment, zinc may be included in amounts ranging from about 7.5 mg to about 22.5 mg. In another specific embodiment, zinc may be included in amounts ranging from about 12 mg to about 18 mg. In another specific embodiment, zinc may be included in amounts ranging from about 13.5 mg to about 16.5 mg. In another embodiment, zinc may be included in an amount of about 15 mg.

In another specific embodiment, zinc may be included in specific ranges or amounts for each specific form. When provided in their specific forms, the provided numerical range or amount includes the amounts of the specific form and/or compounds that are equivalent to the specific form. For example, zinc may be in the form zinc oxide and may be included in the amount of about 15 mg. Accordingly, in this example, “zinc oxide in the amount of about 15 mg” would include 15 mg of zinc oxide and/or its equivalents and would, for example, include a product having 15 mg zinc sulfate instead of zinc oxide.

The compositions, kits and methods of the present invention may comprise or use copper. In a specific embodiment, copper may be included in the forms of a salt, in a chelated form, in a non-chelated form, cupric oxide, copper sulfate, copper gluconate, copper citrate, cupric acetate, alkaline copper carbonate, and copper salicylate.

In another specific embodiment, copper may be included in amounts ranging from about 1.0 mg to about 3.0 mg. In another specific embodiment, copper may be included in amounts ranging from about 1.6 mg to about 2.4 mg. In another specific embodiment, copper may be included in amounts ranging from about 1.8 mg to about 2.2 mg. In another embodiment, copper may be included in an amount of about 2.0 mg.

In another specific embodiment, copper may be included in specific ranges or amounts for each specific form. When provided in their specific forms, the provided numerical range or amount includes the amounts of the specific form and/or compounds that are equivalent to the specific form. For example, copper may be in the form copper oxide and may be included in the amount of about 2.0 mg. Accordingly, in this example, “copper oxide in the amount of about 2.0 mg” would include 2.0 mg of copper oxide and/or its equivalents and would, for example, include a product having 2.0 mg copper sulfate instead of copper oxide.

The compositions, kits and methods of the present invention may comprise or use bioflavanoids. In a specific embodiment of the present invention, bioflavonoids may be included in the form of oligomeric proanthocyanidins (OPCs), epicatechin, genistein, hesperidin, quercetin, rutin, narirutin, naringin, hesperetin, neohesperidin, tangeretin, nobiletin and sinensetin.

In another specific embodiment, bioflavanoids may be included in amounts ranging from about 10 mg to about 200 mg. In another specific embodiment, one or more bioflavanoids may be included a total amount ranging from about 10 mg to about 200 mg.

In another specific embodiment, bioflavanoids may be included in specific ranges or amounts for each specific form. When provided in their specific forms, the provided numerical range or amount includes the amounts of the specific form and/or compounds that are equivalent to the specific form. For example, bioflavanoids may be in the form OPCs and may be included in the amount of about 150 mg. Accordingly, in this example, “OPCs in the amount of about 150 mg” would include 150 mg of OPCs and/or its equivalents and would, for example, include a product having 150 mg epicatechin instead of OPCs.

The compositions, kits and methods of the present invention may comprise or use amino acids. In a specific embodiment, the amino acids may be included in the form of proline, phenylalanine, methionine, threonine, tryptophan, histidine, isoleucine, leucine, asparagine, aspartic acid, glutamic acid, glutamine, serine, tyrosine, valine, lysine, alanine, glycine, tryptophan, cysteine, trimethyl glycine (TMG), L taurine, L-carnitine, acetyl-L-carnitine, N,N-dimethyl glycine and N-acetylcysteine. In another specific embodiment, each amino acid may be included in amounts ranging from about 10 mg to about 100 mg. In another specific embodiment, one or more amino acids may be included in a total amount ranging from about 10 mg to about 100 mg.

In a specific embodiment, the compositions, kits and methods may comprise or use selenium. In another specific embodiment, selenium may be chelated to one or more compounds. In a specific embodiment, the methods and compositions of the present invention may include selenium in a non-chelated form. In a specific embodiment, the methods and compositions of the present invention may include selenium chelated to an amino acid or derivative thereof. In another specific embodiment, the methods and compositions of the present inventions may include a selenium-methionine chelate. In another specific embodiment, selenium may be included in amounts ranging from about 35 μg to about 105 μg. In another specific embodiment, selenium may be included in amounts ranging from about 56 μg to about 84 μg. In another specific embodiment, selenium may be included in amounts ranging from about 63 μg to about 77 μg. In another embodiment, selenium may be included in an amount of about 70 μg.

Specific combinations of various vitamins, nutrients and minerals may be included and utilized in the compositions, kits and methods of the present invention. In one embodiment, vitamins, nutrients or minerals is selected from one or more of the group consisting of vitamin D, iodine, vitamin B9, vitamin B12, and iron. In some embodiments, vitamin D, if present, is in an amount of about 200 I.U. to about 1600 I.U., said iodine, if present, is in an amount of about 100 μg to about 300 μg, said vitamin B9, if present, is in an amount of about 0.2 mg to about 1.5 mg, said vitamin B12, if present, is in an amount of about 2 μg to about 18 μg, said iron, if present, is in an amount of about 13.5 mg to about 40.5 mg.

In a specific embodiment, active ingredients such as twelve carbon chain fatty acid or twelve carbon chain acylglycerols and the vitamins, minerals and nutrients of the present invention, may be included in overages. Adding overages of these compounds may be necessary to meet the amounts claimed on the product label and product insert to ensure that those recited amounts are met throughout the shelf life of the product. Indeed, because of US regulatory requirements that label values reflect minimum contents of these nutrients, deviations in actual nutrient content from label values are usually thought to tend toward overages. Dwyer et al., ANAL BIOANAL CHEM, 389:37-46 (2007). In a specific embodiment, twelve carbon chain fatty acid or twelve carbon chain acylglycerols and one or more of the vitamins, minerals and nutrients may be included in the compositions and methods of the present invention in overages of the recited, specific label amounts of about 100% to about 150% of the label amount, although the overages are dependant on the stability of each ingredient. For example, overages of vitamin D and vitamin B12 may be necessary due to the lack of stability of specific forms. In another example, 5-methyltetrahydrofolate, a form of vitamin B9, is degraded by light, temperature and may degrade during processing and storage. Overages may be larger for some vitamins—particularly those that are less stable and more likely to deteriorate with a long shelf life, those that have other functions (such as antioxidants) in the product itself; for minerals, excess amounts with large overages are probably less likely because of their increased bulk and shelf life stability Dwyer et al., ANAL BIOANAL CHEM, 389:37-46 (2007). Accordingly, when overages are included for any specific active ingredient, at some point in time, these ingredients with overages will degrade so that they fall within the amounts provided in the specific label. Thus, there is no literal difference between the amounts for active ingredients that include overages, and those amounts listed on the specific label. Furthermore, overages provide an equivalent efficacy of the active ingredient over the shelf life of the product. Accordingly, an active ingredient provided in overage amounts is an insubstantial change and performs substantially the same function, in substantially the same way, and leads to substantially the same result as that same active ingredient in the amounts as provided on the specific label.

In another embodiment, the nutritional supplements may include multiple vitamins, nutrients and minerals in one composition. Providing a single composition multivitamin and multinutrient supplement is an appealing feature because it improves patient compliance. Patients, and specifically for example, pregnant patients, often have nausea, and may have difficulties taking multiple pills. A one pill or one composition nutritional supplement that includes the beneficial vitamins, nutrients and minerals in appropriate dosage amounts would thus be beneficial for improving patient compliance in for example, pregnant women. In a specific embodiment of the present invention, the compositions, kits and methods may utilize or administer a composition comprising a twelve carbon chain fatty acid or twelve carbon chain acylglycerol, an omega-3 fatty acid, one or more vitamins, nutrients or minerals and optionally one or more pharmaceutically acceptable carriers. In another embodiment, the compositions, kits and methods may be utilized or administered in swallowable pills, such as a caplet, a tablet and/or a gelcap. In another embodiment, the compositions, kits and methods of the present invention may be utilized or administered in one or more dosage forms selected from the group consisting of a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution.

In another embodiment, the compositions, kits and methods may be utilized or administered in large volume dosage forms. For example, the compositions and kits may be included in large serving volumes or administered in large serving volumes. In a specific embodiment, the serving volumes may be larger than about 2 ml. In another embodiment, the serving volume may be for liquid compositions. In another embodiment, the large dosage forms may be provide in a concentrated powder. If the dosage from is the provided in a concentrated powder, the concentrated powder can be admixed with a liquid by a patient or before administration to a patient. In another specific embodiment, the compositions and kits may be provided as a concentrated powder, already admixed with a liquid. In another specific embodiment, the dosing volumes may be greater than about 50 ml. In a specific embodiment, the dosing volumes may be about 100 ml to about 500 ml. In another specific embodiment, the dosing volumes may be about 100 ml to about 300 ml.

In a specific embodiment, the compositions, kits and methods of the present invention may comprise a twelve carbon chain fatty acid or twelve carbon chain acylglycerol, one or more vitamins, nutrients or minerals, and optionally one or more pharmaceutically acceptable carriers, wherein the composition may comprise dosing volumes greater than about 50 ml. In another embodiment, the compositions, kits and methods of the present invention may comprise additional ingredients that may provide nutritional supplementation or compliance issues appropriate in liquid compositions and/or larger server volumes. In one embodiment, the compositions, kits and methods of the present invention may additionally comprise milk protein concentrate. In another embodiment, the additional ingredient may be selected from one or more of the group consisting of water, sucrose, maltodextrin, milk protein concentrate, soy oil, canola oil, short chain fructooligosaccarides, soy protein isolate, corn syrup, sodium caseinate, and potassium citrate. In another embodiment, the liquid compositions and/or large serving volumes may comprise a natural or artificial flavor selected from one or more of the group consisting of the flavors apple, banana, blueberry, caramel, cherry, chocolate, cinnamon, coffee, cranberry, grape, honey, kiwi, lemon, lime, lemon-lime, mango, mint, orange, peach, pineapple, raspberry, strawberry, tangerine, vanilla, and watermelon.

In another embodiment of the present invention, the active ingredients may comprise a twelve carbon chain fatty acid or twelve carbon chain acylglycerol and multiple vitamins, nutrients and minerals in more than one composition. In a specific embodiment, various twelve carbon chain fatty acids or twelve carbon chain acylglycerols and vitamins, nutrients and minerals may be incorporated into multiple compositions as a kit. In one example, fat soluble compounds such as omega-3 fatty acids, twelve carbon chain fatty acids, and twelve carbon chain acylglycerols, may be included in one composition, whereas water soluble vitamins such as B-complex vitamins and vitamin C may be separated into another composition. In another example, the multiple compositions may be separated due to size or the large dosage amounts of specific ingredients. In another example, the nutritional supplementation of a multivitamin may not be adequate in one composition. Accordingly, all the active ingredients may be divided into a total of two compositions, three compositions, four compositions, five compositions, six compositions and seven compositions. In one embodiment, each composition may have equal amounts of each active ingredient. In another embodiment, compositions may have unequal amounts of various active ingredients, or merely supplemental amounts of specific active ingredients.

In a specific embodiment, the twelve carbon chain fatty acids or twelve carbon chain acylglycerols may be included with various vitamins, minerals and nutrients in multiple compositions as a kit. In a specific embodiment, omega 3 fatty acids such as DHA may be provided in one or more of the compositions of the kit. In a specific embodiment, the kits and the methods of administration thereof, may be a kit comprising a first composition comprising a twelve carbon chain fatty acid or twelve carbon chain acylglycerol, an omega 3 fatty acid, one or more vitamins, nutrients or minerals, and one or more pharmaceutically acceptable carriers; and a second composition comprising a twelve carbon chain fatty acid or twelve carbon chain acylglycerol, an omega-3 fatty acids, one or more vitamins, nutrients or minerals, and one or more pharmaceutically acceptable carriers; wherein the first and second compositions collectively comprise twelve carbon chain fatty acid or twelve carbon chain acylglycerols, omega-3 fatty acids, one or more vitamins, nutrients or minerals, and one or more pharmaceutically acceptable carriers. In one specific embodiment, the kits and methods of administration thereof, may be a kit comprising a first composition with one or more selected from the group consisting of twelve carbon chain fatty acids, twelve carbon chain acylglycerols, omega 3 fatty acids, vitamins, nutrients, and minerals; and optionally one or more pharmaceutically acceptable carriers; and a second composition comprising one or more selected from the group consisting of twelve carbon chain fatty acids, twelve carbon chain acylglycerols, omega 3 fatty acids, vitamins, nutrients, and minerals; and optionally one or more pharmaceutically acceptable carriers; wherein said first and second compositions collectively comprise a twelve carbon chain fatty acid or twelve carbon chain acylglycerol, an omega 3 fatty acid, and one or more vitamins, nutrients or minerals. In another specific embodiment, the kits and the methods of administration thereof, may be a kit comprising two to seven compositions; wherein, two to seven compositions collectively comprise twelve carbon chain fatty acid or twelve carbon chain acylglycerols, one or more vitamins, nutrients or minerals; and one or more inactive ingredients or pharmaceutically acceptable carriers.

In another specific embodiment, the compositions, kits and methods may be utilized or administered for multiple indications or purposes. In one embodiment, the compositions, kits and methods may be utilized or administered as a dietary supplement. In another embodiment, the compositions, kits and methods may be utilized or administered as a prescription prenatal vitamin. In another embodiment, the compositions, kits and methods of the present invention, may be utilized or administered to a patient. The compositions, kits and methods of the present invention, may be utilized or administered to a patient in need of supplementation thereof, for example, twelve carbon chain fatty acid or twelve carbon chain acylglycerol supplementation. In a specific embodiment, the twelve carbon chain fatty acid or twelve carbon chain acylglycerol supplementation may be lauric acid and/or monolaurin. In one embodiment, the compositions, kits and methods of the present invention, may be utilized or administered to a patient as an antiviral, a bactericidal, an antifungal or one or more combinations thereof. In another embodiment, the compositions, kits and methods of the present invention, may be utilized or administered to a patient such as a woman during pregnancy, prenatal or who is breast-feeding. In another embodiment, the compositions, kits and methods of the present invention may be utilized or administered, once a day, twice a day, three times a day, four times a day and five times a day. When multiple compositions are provided in a kit, the compositions may be co-administered at the same or administered separately.

In another embodiment, the compositions, kits and methods of the present invention may be used or utilized in one or more dosage forms. In a specific embodiment, the kits may comprise multiple compositions utilizing multiple dosage forms. The ingredients of the present invention may thus be combined into a composition which may be in the a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution, and which may be administered alone or in suitable combination with other components. For example, the compositions and kits of the present invention may be utilized and administered in one or more caplets or gel caps as practical for ease of administration. Each of the vitamins, nutrients and minerals is commercially available, and can be blended to form a single composition or can form multiple compositions, which may be co-administered.

To prepare the compositions of the present invention, each of the active ingredients may be combined in intimate admixture with a suitable carrier according to conventional compounding techniques. The carrier may take a wide variety of forms depending upon the form of the preparation desired for administration, e.g., oral, sublingual, nasal, topical patch, or parenteral.

In preparing the composition in oral dosage form, any of the usual media may be utilized. For liquid preparations (e.g., suspensions, elixirs, and solutions), media containing, for example water, oils, alcohols, flavoring agents, preservatives, coloring agents and the like may be used. Pharmaceutically acceptable carriers such as starches, sugars, diluents, granulating agents, lubricants, binders, disintegrating agents and the like may be used to prepare oral solids (e.g., powders, caplets, pills, tablets, capsules, and lozenges). Controlled release forms may also be used. Because of their ease in administration, caplets, tablets, pills, and capsules represent the most advantageous oral dosage unit form, in which case solid carriers are employed. If desired, tablets may be sugar coated or enteric coated by standard techniques. All of these pharmaceutical carriers and formulations are well known to those of ordinary skill in the art. See, e.g., WADE & WALLER, HANDBOOK OF PHARMACEUTICAL EXCIPIENTS (2nd ed. 1994).

In a specific embodiment, the present invention may comprise kits or compositions in the dosage form of a soft-gel gelcap. A soft-gel may be a one-piece, sealed, soft gelatin shell that contains a solution, a suspension, or a semi-solid paste. Soft-gels can be used to contain liquids wherein the active ingredients are present in the dissolved or suspended state. Soft-gels have been widely known and used for many years and for a variety of purposes. Because soft-gels have properties that are quite different from two-piece, hard shell capsules, the soft-gels are capable of retaining a liquid fill material. Soft-gels are often used to encapsulate consumable materials, including vitamins, dietary supplements, pharmaceuticals, and the like, in a liquid vehicle or carrier. Soft-gels are a unique dosage form that can provide distinct advantages over more traditional dosage forms such as tablets, hard-shell capsules, and liquids. These advantages include patient compliance and consumer preference, improved bioavailability, speed of product development in many cases, shortened manufacturing time, enhanced drug stability due to less exposure of the active ingredient to oxygen, excellent dose uniformity, and product differentiation.

In a specific mode of administration, the dosage forms, may be swallowable, chewable or dissolvable. Swallowable compositions are well known in the art and are those that do not readily dissolve when placed in the mouth and may be swallowed whole without any chewing or discomfort. In a specific embodiment of the present invention the swallowable compositions may have a shape containing no sharp edges and a smooth, uniform and substantially bubble free outer coating.

To prepare the swallowable compositions of the present invention, each of the active ingredients may be combined in intimate admixture with a suitable carrier according to conventional compounding techniques. In a specific embodiment of the swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia Pharmaceutical carriers and formulations for swallowable compounds are well known to those of ordinary skill in the art. See generally, e.g., WADE & WALLER, HANDBOOK OF PHARMACEUTICAL EXCIPIENTS (2nd ed. 1994).

Chewable compositions are those that have a palatable taste and mouthfeel, are relatively soft and quickly break into smaller pieces and begin to dissolve after chewing such that they are swallowed substantially as a solution.

In order to create chewable compositions, certain ingredients should be included to achieve the attributes just described. For example, chewable compositions should include ingredients that create pleasant flavor and mouthfeel and promote relative softness and dissolvability in the mouth. The following discussion describes ingredients that may help to achieve these characteristics.

Chewable compositions preferably have a pleasant or palatable flavor and a pleasant mouthfeel. A variety of ingredients can be included in the compositions of the present invention to enhance mouthfeel.

In the chewable compositions of the present invention, sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Further, by way of example and without limitation, fondant or gums such as gelatin, agar, arabic gum, guar gum, and carrageenan may be added to improve the chewiness of the compositions. Fatty materials that may be included in the present invention include, by way of example and without limitation, vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used in the present invention to enhance the texture, the mouthfeel, or both of the chewable nutritional supplement compositions described herein. By “enhance the texture” it is meant that the alkyl polysiloxane improves one or more of the stiffness, the brittleness, and the chewiness of the chewable supplement, relative to the same preparation lacking the alkyl polysiloxane. By “enhance the mouthfeel” it is meant that the alkyl polysiloxane reduces the gritty texture of the supplement once it has liquefied in the mouth, relative to the same preparation lacking the alkyl polysiloxane.

Alkyl polysiloxanes generally comprise a silicon and oxygen-containing polymeric backbone with one or more alkyl groups pending from the silicon atoms of the back bone. Depending upon their grade, they can further comprise silica gel. Alkyl polysiloxanes are generally viscous oils. Exemplary alkyl polysiloxanes that can be used in the swallowable, chewable or dissolvable compositions of the present invention include, by way of example and without limitation, monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than about 90.5% by weight (CH₃)₃—Si{OSi(CH₃)₂}CH₃ in admixture with about 4.0% to about 7.0% by weight SiO₂.

Chewable compositions should begin to break and dissolve in the mouth shortly after chewing begins such that the compositions can be swallowed substantially as a solution. The dissolution profile of chewable compositions may be enhanced by including rapidly water-soluble fillers and excipients. Rapidly water-soluble fillers and excipients preferably dissolve within about 60 seconds of being wetted with saliva. Indeed, it is contemplated that if enough water-soluble excipients are included in the compositions of the present invention, they may become dissolvable rather than chewable composition forms. Examples of rapidly water soluble fillers suitable for use with the present invention include, by way of example and without limitation, saccharides, amino acids and the like. Disintegrants also may be included in the compositions of the present invention in order to facilitate dissolution. Disentegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include, by way of example and without limitation, starches, such as corn starch, potato starch, pre-gelatinized and modified starches thereof, cellulosic agents, such as Ac-di-sol, montrnorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable, chewable and/or dissolvable compositions of the present invention so long as they are consistent with the objectives described herein. For example, binders, are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl futmarate, and others known to those of ordinary skill in the art.

Glidants improve the flow of powder blends during manufacturing and minimize composition weight variation. Glidants that may be used in the present invention include, by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art Coloring agents also can include pigments, dyes, tints, titanium dioxide, natural coloring agents, such as grape skin extract, beet red powder, beta carotene, annato, carmine, turneric, paprika and others known to those of ordinary skill in the art. It is recognized that no colorant is required in the nutritional supplement compositions described herein.

If desired, the compositions of the present invention may be sugar coated or enteric coated by standard techniques. The unit dose forms may be individually wrapped, packaged as multiple units on paper strips or in vials of any size, without limitation. The swallowable, chewable or dissolvable compositions of the present invention may be packaged in unit dose, rolls, bulk bottles, blister packs and combinations thereof, without limitation.

The swallowable, chewable or dissolvable compositions of the present invention may be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable nutritional supplements with improved mouthfeel. U.S. Pat. No. 5,965,162, relates to kits and methods for preparing multi-vitamin comestible units which disintegrate quickly in the mouth, especially when chewed. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in WADE & WALLER, HANDBOOK OF PHARMACEUTICAL EXCIPIENTS (2nd ed. 1994). Inactive ingredients, fillers and excipients are commercially available from companies such as Aldrich Chemical Co., FMC Corp, Bayer, BASF, Alexi Fres, Witco, Mallinckrodt, Rhodia, ISP, and others.

A specific embodiment of the present invention may comprise kits or swallowable compositions packaged in blister packs. Blister packs as packaging for swallowable compositions are well known to those of ordinary skill in the art. Blister packs may be made of a transparent plastic sheet which as been formed to carry a matrix of depression or blisters. One or more swallowable compositions are received in each depression or blister. A foil or plastic backing is then adhered across the plane of the sheet sealing the swallowable compositions in their respective blisters. Examples of materials used for the blister packs include, but are not limited to, aluminum, paper, polyester, PVC, and polypropylene. Alternative materials are known to those of ordinary skill in the art. To remove a swallowable composition, the depression material is pressed in and the composition is pushed through the backing material. Multiple blister packs may be placed in an outer package, often a box or carton for sale and distribution.

Another specific embodiment of the present invention may comprise kits or swallowable compositions packaged in bottles. The bottle may be glass or plastic in form with a pop or screw top cap. Bottle packaging for compositions in swallowable form are well known to those of ordinary skill in the art.

Additionally, the unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation was prepared in gel-cap form, including the appropriate excipients, by standard methods known to those of ordinary skill in the art:

| | |
|---|---|
| Lauric Acid | 60 mg |
| DHA | 200 mg |

### Example 2

A composition of the following formulation was prepared in gel-cap form, including the appropriate excipients, by standard methods known to those of ordinary skill in the art. In this example, the major source of lauric acid is from coconut oil and the source of DHA is from algae oil. Minimal amounts of lauric acid are also provided in the algae oil.

| | |
|---|---|
| Lauric Acid | 60 mg |
| DHA | 200 mg |

### Example 3

A composition of the following formulation was prepared in gel-cap form, including the appropriate excipients, by standard methods known to those of ordinary skill in the art. In this example, the major source of lauric acid is from coconut oil and the source of DHA is from fish oil. Minimal amounts of lauric acid are also provided in the fish oil.

| | | |
|---|---|---|
| Lauric Acid | 60 | mg |
| DHA | 200 | mg |
| EPA | 24 | mg |
| Vitamin D (Vitamin D3) | 1000 | IU |
| Vitamin B9 (Folic Acid) | 1 | mg |
| Vitamin B12 | 12 | μg |
| Iron | 27 | mg |
| Iodine | 200 | μg |

### Example 4

A composition of the following formulation was prepared in gel-cap form, including the appropriate excipients, by standard methods known to those of ordinary skill in the art. In this example, the major source of lauric acid is from coconut oil and the source of DHA is from algae oil. Minimal amounts of lauric acid are also provided in the algae oil:

| | | |
|---|---|---|
| Vitamin A (Beta Carotene) | 1100 | IU |
| Vitamin C | 12 | mg |
| Vitamin D (Vitamin D3) | 1000 | IU |
| Vitamin E | 10 | IU |
| Vitamin B1 | 1.6 | mg |
| Vitamin B2 (Riboflavin) | 1.8 | mg |
| Vitamin B3 (Niacinamide) | 15 | mg |
| Vitamin B6 | 2.5 | mg |
| Vitamin B9 (Folic Acid) | 1 | mg |
| Vitamin B12 | 12 | μg |
| Iron | 27 | mg |
| Magnesium | 5 | mg |
| Zinc | 15 | mg |
| Copper | 2.0 | mg |
| Iodine | 200 | μg |
| Lauric acid | 60 | mg |
| DHA | 200 | mg |

### Example 5

A composition of the following formulation was prepared in gel-cap form, including the appropriate excipients, by standard methods known to those of ordinary skill in the art:

| | | |
|---|---|---|
| Vitamin A (acetate) | 1100 | IU |
| Beta Carotene | 600 | IU |
| Vitamin B₁ (thiamine mononitrate) | 1.6 | mg |
| Vitamin B₂ (riboflavin) | 1.8 | mg |
| Vitamin B₃ (niacinamide) | 15 | mg |
| Vitamin B₆ (pyridoxine hydrochloride) | 2.5 | mg |
| Vitamin B₉ (folic acid) | 1000 | μg |
| Vitamin B₁₂ (cyanocobalamin) | 5 | μg |
| Vitamin C (ascorbic acid) | 60 | mg |
| Vitamin D (cholecalciferol) | 400 | IU |
| Vitamin E (d-alpha-tocopheryl acetate) | 30 | IU |
| Iron (polysaccharide complex) | 29 | mg |
| Magnesium (magnesium oxide) | 25 | mg |
| Zinc (zinc oxide) | 15 | mg |

A second composition of the following formulation is prepared in gel-cap form by standard methods known to those of ordinary skill in the art:

| | |
|---|---|
| Lauric acid | 60 mg |
| DHA | 200 mg |

### Example 6

In another example, various active ingredients may be divided into multiple compositions or a kit. In this non limiting example, a first composition of the following formulation is prepared in gel-cap form, including the appropriate excipients, by standard methods known to those of ordinary skill in the art:

| | | |
|---|---|---|
| Vitamin A (Beta Carotene) | 550 | IU |
| Vitamin C | 6 | mg |
| Vitamin D (Vitamin D3) | 500 | IU |
| Vitamin E | 5 | IU |
| Vitamin B1 | 0.8 | mg |
| Vitamin B2 (Riboflavin) | 0.9 | mg |
| Vitamin B3 (Niacinamide) | 7.5 | mg |
| Vitamin B6 | 1.25 | mg |
| Vitamin B9 (Folic Acid) | 0.1 | mg |
| Vitamin B12 | 6.0 | μg |
| Iron | 13.5 | mg |
| Magnesium | 2.5 | mg |
| Zinc | 7.5 | mg |
| Copper | 1.0 | mg |
| Iodine | 100 | μg |
| Monolaurin | 30 | mg |
| DHA | 100 | mg |

A second composition of the following formulation is prepared in gel-cap form by standard methods known to those of ordinary skill in the art:

| | | |
|---|---|---|
| Vitamin A (Beta Carotene) | 550 | IU |
| Vitamin C | 6 | mg |
| Vitamin D (Vitamin D3) | 500 | IU |
| Vitamin E | 5 | IU |
| Vitamin B1 | 0.8 | mg |
| Vitamin B2 (Riboflavin) | 0.9 | mg |
| Vitamin B3 (Niacinamide) | 7.5 | mg |
| Vitamin B6 | 1.25 | mg |
| Vitamin B9 (Folic Acid) | 0.1 | mg |
| Vitamin B12 | 6.0 | μg |
| Iron | 13.5 | mg |
| Magnesium | 2.5 | mg |
| Zinc | 7.5 | mg |
| Copper | 1.0 | mg |
| Iodine | 100 | μg |
| Monolaurin | 30 | mg |
| DHA | 100 | mg |

### Example 7

In this non limiting example, a first composition of the following formulation is prepared in gel-cap form, including the appropriate excipients, by standard methods known to those of ordinary skill in the art:

| | | |
|---|---|---|
| Vitamin A (Beta Carotene) | 4000 | IU |
| Vitamin C | 60 | mg |
| Vitamin D (Vitamin D3) | 400 | IU |
| Vitamin E | 30 | IU |
| Vitamin B1 | 1.7 | mg |
| Vitamin B2 (Riboflavin) | 2.0 | mg |
| Vitamin B3 (Niacinamide) | 20 | mg |
| Vitamin B6 | 2.5 | mg |
| Vitamin B9 (Folic Acid) | 0.8 | mg |
| Vitamin B12 | 8 | μg |
| Vitamin B7 (biotin) | 300 | μg |
| Pantothenic acid | 10 | mg |
| Calcium (elemental) | 300 | mg |
| Iron | 28 | mg |
| Magnesium | 50 | mg |
| Zinc | 15 | mg |
| Copper | 2.0 | mg |
| Iodine | 150 | μg |
| Lauric Acid | 60 | mg |

A second composition of the following formulation is prepared in gel-cap form by standard methods known to those of ordinary skill in the art:

| | |
|---|---|
| DHA | 200 mg |
| EPA | 24 mg |

### Example 8

In this non limiting example, active ingredients may be separated into various compositions as a kit. In this non limiting example, a first composition of the following formulation is prepared in gel-cap form, including the appropriate excipients, by standard methods known to those of ordinary skill in the art:

| | | |
|---|---|---|
| Vitamin A (Beta Carotene) | 4000 | IU |
| Vitamin C | 100 | mg |
| Vitamin D (Vitamin D3) | 400 | IU |
| Vitamin E | 11 | IU |
| Vitamin B1 | 1.5 | mg |
| Vitamin B2 (Riboflavin) | 1.7 | mg |
| Vitamin B3 (Niacinamide) | 18 | mg |
| Vitamin B6 | 2.6 | mg |
| Vitamin B9 (Folic Acid) | 0.8 | mg |
| Vitamin B12 | 4 | μg |
| Calcium | 150 | mg |
| Iron | 27 | mg |
| Zinc | 25 | mg |
| Lauric Acid | 60 | mg |

A second composition of the following formulation is prepared in gel-cap form by standard methods known to those of ordinary skill in the art:

| | |
|---|---|
| DHA | 200 mg |
| EPA | 24 mg |

### Example 9

In this non limiting example, active ingredients may be separated into various compositions in a kit. In this non limiting example, a first composition of the following kit is prepared in a tablet form, including the appropriate excipients, by standard methods known to those of ordinary skill in the art:

| | | |
|---|---|---|
| Vitamin A (Beta Carotene) | 2700 | IU |
| Vitamin C (Ascorbic Acid) | 70 | mg |
| Vitamin D (Cholecalciferol) | 400 | IU |
| Vitamin E (as dl alpha tocopheryl acetate) | 30 | IU |
| Folic Acid | 1 | mg |
| Vitamin B1 (Thiamine Mononitrate) | 1.6 | mg |
| Vitamin B2 (Riboflavin) | 1.8 | mg |
| Vitamin B6 (Pyridoxine Hydrochloride) | 2.5 | mg |
| Vitamin B12 (Cyanocobalamin) | 12 | μg |
| Niacin (as Niacinamide) | 18 | mg |
| Calcium (Calcium Carbonate) | 100 | mg |
| Elemental Iron (Ferrous Fumarate) | 65 | mg |
| Magnesium (Magnesium Oxide) | 25 | mg |
| Zinc (Zinc Oxide) | 25 | mg |
| Copper (Copper Oxide) | 2 | mg |

A second composition of the following kit is prepared in gel-cap form by standard methods known to those of ordinary skill in the art. In this example, the major source of lauric acid is from coconut oil, and the source of DHA and EPA is from fish oil. Minimal amounts of lauric acid are also provided in the fish oil:

| | |
|---|---|
| DHA | 200 mg |
| Lauric Acid | 60 mg |
| EPA | 24 mg |
| Iodine | 200 μg |

### Example 10

A study is undertaken testing the fatty acid composition in women's milk after nutritional supplementation with fatty acids including lauric acid. Fifty women will partake in the study. Lactating women aged 19-43 y participate in the study during the first 6 months of lactation. The women remain in the study for about 10 weeks including a 2-wk washout period between consumption of the test fatty acids. The diets of the subjects remain constant during the study. The Diet Habit Survey indicates that there is no significant differences in cholesterol-saturated fat, carbohydrate, fish, or total scores. Subjects consume the coconut oil composition once in 2-wk intervals. A 40 g coconut oil composition is consumed in the morning by each patient in place of breakfast. Subjects collect milk samples once in the morning before consuming the test formula; once at each of the following time points after consuming the formula: 6, 10, 14, and 24 h; and once daily for 4-7 days. The results of this study show an acute response, especially within the first 24 hr. Ingestion of the formula containing 40 g coconut oil increases the milk content of lauric acid over time (P<0.001). Lauric acid is increased from 3.9% of fatty acids at baseline to 9.2% at 10 h and 9.6% at 14 hr. See Francois et al., AM J CLIN NUTR, 67(2): 301-8 (1998).

### Example 11

Fourteen lactating women drink two test formulas, each containing a different fat: menhaden oil, and coconut oil. The subjects collect a midfeeding milk sample before consuming the breakfast test formula and additional samples at 6, 10, 14, and 24 h and then once daily for 4-7 d. Fatty acids of special interest included eicosapentaenoic and docosahexaenoic acids from menhaden oil, and lauric acid from coconut oil. Each of these fatty acids increases significantly in human milk within 6 hrs of consumption of the test formulas (P<0.001). Maximum increases occur 14 hrs after coconut oil (lauric acid), and menhaden oil (eicosapentaenoic acid); and 24 hrs after menhaden oil (docosahexaenoic acid). Surprisingly, lauric acid remains significantly elevated in milk (P<0.05) for only about 10-24 hrs, whereas docosahexaenoic acid and eicosapentaenoic acid remain significantly elevated in milk for 2 days and three days respectively. These data support the hypothesis that there is a rapid transfer of dietary fatty acids from chylomicrons into human milk. These data also surprisingly support that lauric acid significantly increases in milk for only up to about 24 hr. Accordingly, a daily source or daily supplementation of lauric acid for a lactating patient to retain a high level of lauric acid in human milk is suggested.

### Example 12

A study is undertaken to evaluate the effectiveness of the compositions of the present invention in the treatment of patients. The objective of the study is to determine whether oral intake of the compositions results in an improvement of the nutritional status of patients with regard to the specific vitamins and minerals contained in the administered compositions.

A double-blind, placebo controlled study is conducted over a six-month period. A total of 120 subjects (60 pregnant women entering the second trimester of pregnancy and 60 lactating women), aged 20-35 years, are chosen for the study. An initial assessment of the nutritional status of each woman is conducted. Vitamin A and vitamin B6 are measured using high performance liquid chromatography. Erythrocyte transketolase activity is used to measure vitamin B1 levels. Vitamin B2 levels are determined by assessment of erythrocyte glutathione reductase activity. Vitamin B3 levels are assessed by measuring urinary excretion of N′ methylnicotinamide and its pyridone. Vitamin B9 is measured by radioimmunoassay (RIA), specifically The Solid Phase No Biol Folic Acid Kit (Diagnostic Products, Los Angeles, Calif.). Vitamin B12 is measured by RIA using human intrinsic factor as a binder. Vitamin C levels are measured by spectrophotometric and colorimetric methods. Vitamin D is measured using an extraction double-antibody RIA (Dia Sorin, Inc., Stillwater, Minn.). The peroxide hemolysis test is used to determine vitamin E status. Iron levels are measured using standard spectrophotometry. Iodine levels are measured by HPLC. Lauric acid levels and other fatty acids and acylglycerols are measured by reverse phase HPLC. Magnesium levels are measured by absorbance of a magnesium chelate with xylidl blue at 660 nM. Zinc levels are assessed using flame atomic absorption spectrometry (Perkins Elmer 460, Norwalk, Conn.). DHA is measured and quantified using gas chromatography procedures.

Additionally, total serum homocysteine levels are determined by extraction on the Multi-Prep® gravity series GVSA-100 column, a strong anion exchange gravity flow column, and measurement by gas chromatography/mass spectrometry. Biochemical Diagnostics, Austin, Tex.

The 120 subjects are separated into four separate groups of 30 women. In a first group comprising only pregnant women and in a second group comprising only lactating women, each subject is administered one dosage form of the composition as described in Example 3 twice a day. In a third group comprising only pregnant women and in a fourth group comprising only lactating women, each subject is administered one placebo dosage form twice a day. Thus, dosage form administration occurs every 12 hours. No other nutritional supplements are taken by the subjects during the assessment period.

An assessment of the nutritional status of each woman is conducted utilizing the methods described above at one month intervals for a six month period. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1, 2, 3, 4, 5, and 6 months, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status of vitamin, mineral, and nutrient levels measured is observed in the treated subjects over the controls upon completion of the study. Homocysteine levels in women receiving supplements remain unelevated. Therefore, the study confirms that oral administration of the compositions of the present invention is effective in improving the nutritional status of patients. The length of gestation is increased by approximately six days in women receiving supplements, due to DHA intake, and their homocysteine levels are not elevated, due to folic acid intake, leading to a better prognosis regarding risk of neural tube defects in their infants.

While specific embodiments of the present invention have been described, other and further modifications and changes may be made without departing from the spirit of the invention. All further and other modifications and changes are included that come within the scope of the invention as set forth in the claims. The disclosure of each publication cited above is expressly incorporated by reference in its entirety to the same extent as if each were incorporated by reference individually.

1. A composition comprising lauric acid, DHA, one or more vitamins, nutrients or minerals, and one or more pharmaceutically acceptable carriers, for nutritional supplementation in a patient.

2. (canceled)

3. (canceled)

4. The composition of claim 1, wherein said lauric acid is derived from a natural source, is synthesized, or is genetically modified or engineered.

5. The composition of claim 4, wherein said natural source of said lauric acid is selected from one or more of the group consisting of algae oil, coconut oil, babassu palm oil, palm kernel oil, ouricuri oil, tucum oil, muru-muru oil, jaboty kernel oil, khakan kernel oil, dika nut oil, ucuhuba butter, cinnamon oil, Brachyandra Calophylla seed oil, California laurel seed oil, camphor kernel fat, cohune nut oil, Cuphea seed oil, Heteranthus epilobiifolia seed oil, Irvingia Gabonesis Kernel Fat, jack bean oil, Khakan fat, Pelu fat, Laurel Berry oil, Lindera Umbellata seed oil, Ouricouri Tallow, Pindo Palm kernel oil, Pisa oil, Spicebush Kernel fat, Tucum Kernel oil, cuphea plants, Lauraceae plants, Umbelliferae plants, Arecaceae palms, Lythraceae plants, fruits of Laurus nobilis, qualea plants, Actinodaphne seed oil, cohune palms, Pycanthus kombo, Virola surinamensis, peach palm seed, betel nut, date palm seed, macadamia nut, plum, Watermelon seed, Citrullus lanatus, and Pumpkin flower.

6. The composition of claim 4, wherein said natural source of said lauric acid is selected from one or more of the group consisting of coconut oil, palm kernel oil, babassu oil and algae oil.

7. The composition of claim 1, wherein said lauric acid is present in an amount greater than about 20 mg.

8. The composition of claim 1, wherein said lauric acid is present in an amount of about 30 mg to about 10 g.

9. The composition of claim 1, wherein said lauric acid is present in an amount of about 30 mg to about 280 mg.

10. The composition of claim 1, wherein said lauric acid is present in an amount of about 60 mg.

11-14. (canceled)

15. The composition of claim 1, wherein said vitamins, nutrients or minerals is selected from one or more of the group consisting of vitamin D, iodine, vitamin B1, vitamin B2, vitamin B3, vitamin B6, vitamin B7, vitamin B9, vitamin B12, vitamin E, vitamin A, vitamin C, iron, zinc, copper, magnesium, calcium, molybdenum, chlorine, vitamin K, manganese, selenium, choline, phosphorous, bioflavonoids, CoQ10, chromium, alpha lipoic acid, lutein and antioxidants.

16-17. (canceled)

18. The composition of claim 1, wherein the source of said DHA is one or more of the group consisting of animal, fish, plants, algae, microorganism production and production by genetic engineering.

19. The composition of claim 18, wherein the source of said DHA is fish oil.

20. The composition of claim 18, wherein the source of said DHA is algae oil.

21. The composition of claim 1, wherein said DHA is present in an amount of about 100 mg to about 300 mg.

22. The composition of claim 1, wherein said DHA is present in an amount of about 180 mg to about 220 mg.

23. The composition of claim 1, wherein said DHA is present in an amount of about 200 mg.

24. The composition of claim 1, wherein the source of said lauric acid is selected from one or more of the group consisting of fish oil, algae oil and coconut oil.

25. The composition of claim 24, wherein the source of said lauric acid is said algae oil and coconut oil.

26. The composition of claim 25, wherein said lauric acid is present in an amount of about 5 mg to about 30 mg from said algae oil and about 25 mg to about 250 mg from said coconut oil.

27. The composition of claim 25, wherein said lauric acid is present in an amount of about 5 mg to about 25 mg from said algae oil and about 25 mg to about 100 mg from said coconut oil.

28. The composition of claim 25, wherein said lauric acid is present in an amount of about 10 mg from said algae oil and about 50 mg from said coconut oil.

29. The composition of claim 25, wherein said lauric acid is present in an amount of about 5 mg to about 30 mg from said algae oil and about 25 mg to about 250 mg from said coconut oil and said DHA is present in an amount of about 100 mg to about 300 mg.

30. The composition of claim 25, wherein said lauric acid is present in an amount of about 10 mg from said algae oil and 50 mg from said coconut oil and said DHA is present in an amount of about 200 mg.

31. The composition of claim 25, wherein said coconut oil is present in an amount of about 50 mg to about 500 mg and said algae oil is present in an amount of about 200 mg to about 600 mg.

32. The composition of claim 1, wherein said composition is a dietary supplement.

33. The composition of claim 1, wherein said composition is a prescription prenatal supplement.

34. The composition of claim 1, wherein said composition is a prescription supplement for women during pregnancy.

35. The composition of claim 1, wherein said composition is a prescription postnatal supplement.

36. The composition of claim 1, wherein said composition is a prescription drug.

37. The composition of claim 1, wherein said composition is in a dosage form selected from the group consisting of a capsule, tablet, caplet, gel caplet (gelcap), syrup, a liquid composition, a powder, a concentrated powder, a concentrated powder admixed with a liquid, a chewable form, a swallowable form, a dissolvable form, an effervescent, a granulated form, and an oral liquid solution.

38. The composition of claim 37, wherein said dosage form is a gelcap.

39. A method comprising administering the composition of claim 1 to a patient.

40. The method of claim 39, wherein the patient is in need of lauric acid supplementation.

41. The method of claim 39, wherein said patient is in need of said lauric acid as an antiviral, a bactericidal, an antifungal or one or more combinations thereof.

42. The method of claim 39, wherein said patient is human.

43. The method of claim 42, wherein said human is a woman.

44. The method of claim 43, wherein said woman is pregnant.

45. The method of claim 43, wherein said woman is prenatal.

46. The method of claim 43, wherein said woman is breast feeding.

47-113. (canceled)
